(19) United States
(12) Patent Application Publication
(10) Pub. No.: US 2003/0054355 A1
(43) Pub. Date: Mar. 20, 2003

(54) Microsensors and method for detecting target analytes

(76) Inventor: Peter Warthoe, Copenhagen (DK)

Correspondence Address: FLEHR HOHBACH TEST, ALBRITTON & HERBERT LLP, Suite 3400, Four Embarcadero Center, San Francisco, CA, 94111-4187, US

(21) Appl. No.: 09/944,161

(22) Filed: Aug. 30, 2001

Related U.S. Application Data
(60) Provisional application No. 60/261,222, filed on Jan. 12, 2001.

(30) Foreign Application Priority Data
Sep. 4, 2000 (DK) PA 2000 01310

(51) Int. Cl. C12Q 1/68; C12M 1/34

(52) U.S. Cl. 435/6; 435/287.2

(57) ABSTRACT

The present invention relates generally to methods and compositions for analyzing binding molecules including proteins and nucleic acid-molecules. In addition the invention relates to the use of microsensors that rely on non-fluorescent detection system consisting of a sensor using microscopic flexible mechanical structures such as micro-cantilevers or micro-membranes integrated into a microscopic chambers for detection of a wide variety of biological-based assays.

This application claims priority to Danish patent application PA 2000 01310, filed Sep. 4, 2000, and U.S. Provisional application serial number 60/261,222, filed Jan. 12, 2001, both of which are expressly incorporated herein by reference.

### FIELD OF THE INVENTION

The present invention relates generally to methods, compositions and devices for analyzing molecules including proteins and nucleic acid molecules. The invention relates to the use of microsensors that rely on detection system comprising a microsensor such as a microcantilever or micromembrane integrated into a microscopic chamber to detect such molecules.

### BACKGROUND OF THE INVENTION

Detection and analysis of biological molecules including nucleic acid molecules are among the most important techniques in biology. They are at the heart of molecular biology and play a rapidly expanding role in the rest of biology. A number of methods have been developed which permit the implementation of extremely sensitive assays based on nucleic acid detection. Most of these methods employ exponential amplification of targets or probes. These include the polymerase chain reaction (PCR), ligase chain reaction (LCR), self-sustained sequence replication (3SR), nucleic acid sequence based amplification (NASBA), strand displacement amplification (SDA), and amplification with Q.beta. replicase (Birkenmeyer and Mushahwar, J. Virological Methods, 35:117-126 (1991); Landegren, Trends Genetics, 9:199-202 (1993)) and Rolling Circle Amplification, RCA (Landegren U, Nucleic-Acids Res. Nov 15, 1998 :26(22):5073-8).

If the analysis of nucleic acid molecules is to continue being useful in practical diagnostic applications it is desirable to assay for many targets simultaneously. Such multiplex assays are typically used to detect five or more targets. It is also desirable to obtain accurate quantitative data for the targets in these assays. In a multiplex assay, it is especially desirable that quantitative measurements of different targets accurately reflect the true ratio of the target sequences.

Generally, following essentially all biochemical reactions, analysis entails some form of detection step. Of special interest is the detection of nucleic acid hybridizations and antibody-antigen binding reactions. Ideally, detection should be sensitive. It should allow processing of multiple samples and should not include any form for modification of the biological material. In addition, it should be quite easy and fast to use at routine basis. The last two requirement are particularly important if the technology should be widespread including locations where advanced molecular biology equipment are not available e.g. a medical doctor practice or bio-clinical laboratory for routine molecular diagnostics blood testing. However, current detection techniques are somewhat limited in these characteristics.

Hybridization of nucleic acid molecules is generally detected by autoradiography or phosphor image analysis when the hybridization probe contains a radioactive label or by densitometer when the hybridization probe contains a label, such as biotin or digoxin, that is recognized by an enzyme-coupled antibody or ligand.

When a radiolabeled probe is used, detection by autoradiography suffers from film limitations, such as reciprocity failure and non-linearity. These film limitations can be overcome by detecting the label by phosphor image analysis. However, radiolabels have safety requirements, increasing resource utilization and necessitating specialized equipment and personnel training. For such reasons, the use of nonradioactive labels has been increasing in popularity. In such systems, nucleotides contain a label, such as biotin or digoxin, which can be detected by an antibody or other molecule that is labeled with an enzyme reactive with a chromogenic substrate. Alternatively, fluorescent labels may be used. These systems do not have the safety concerns as described above, but use components that are often labile and may yield nonspecific reactions, resulting in high background (i.e., low signal-to-noise ratio). One major disadvantage of the above described labeling methods is the need for modification of the biological material. This makes them not very attractive outside high specialized genetics laboratories.

Antibody-antigen binding reactions may be detected by one of several procedures. As for nucleic acid hybridization, a label, radioactive or nonradioactive, is typically conjugated to the antibody. The types of labels are similar: enzyme reacting with a chromogenic substrate, fluorescent, hapten that is detected by a ligand or another antibody, and the like. As in detection of nucleic acid hybridization, similar limitations are inherent in these detection methods. In general all detection methods known today require at modification step of the molecule e.g DNA or RNA or protein that should be detected. This makes the current detection methods very work demanding and in general not very user friendly since many steps are required before the final result are obtained.

The polymerase chain reaction (PCR) is a method for specific amplification of DNA fragments. The simplicity and high efficiency of the reaction makes it not only a very powerful research method, but also a very reliable and sensitive diagnostic tool for trite detection of nucleic acids of different pathogens. The PCR has been utilized many times in the diagnosis of numerous diseases. However, this reaction, although efficient and simple has not found a substantial niche in the diagnostic laboratories around the world. The basic PCR techniques are described in U.S. Pat. No. 4,683,195 and 4,683,202 to Mullis, et al., the disclosures of which are incorporated herein. While these techniques have found widespread use in biology, their usefulness in clinical applications has been principally limited by three factors, to wit: (1) conventional PCR does not yield quantitative data it because the amount of nucleic acid increases exponentially and plateaus; (2) it will occasionally amplify nonspecific nucleic acids, and (3) the PCR products must be assessed by semi-quantitative methods such as Southern blotting and densitometry. As a result, most PCR assays are limited to use in applications where the presence or absence of a specific, known nucleic acid molecule (usually DNA) is to be determined.

Researchers have developed various methods intended to allow for quantification of PCR-amplified DNA or RNA. Generally, these approaches involve amplification followed by size analysis on agarose gels or DNA/RNA hybridizations followed by isotopic or enzymatic detection. For example, in Proc. Ntl. Acad. Sci. USA, (1992) 89:3241-3245, a method was reported involving heat (rather than alkaline) denaturation of the PCR product and hybridization in solution of the separated strands to two oligonucleotide probes. One probe is biotin labeled (a “capture” probe); the other is labeled with horseradish peroxidase (HRP) (a “detector” probe). Solution hybridization of the PCR product strands to the probes is performed in microtiter plate wells. These plate wells are coated with streptavidin hydrophobically bound thereto which is intended to bind with the biotinylated probe. After washing, an HRP chromogen is added to the wells, absorbance is measured by a microtiter plate reader and ratios of PCR product separately bound by the probes are measured against a standard curve. One major reason of this delayed acceptance of the PCR in practical diagnostics is inefficient methods for the detection of the PCR products. The most common way of detection is agarose gel electrophoresis. This method requires relatively large amounts of the amplified DNA. To obtain this large amount of DNA the PCR is usually carried out through many cycles of amplification, which makes the reaction very sensitive to cross-contamination of treated specimens, or increases non-specific products.

These non-specific products can lead to misinterpretation of the results. In addition, gel electrophoresis detection of PCR products is not amenable to the needs of routine diagnostic laboratories, which are unlikely to have appropriate equipment. PCR results are generally interpreted by visual analysis of a band stained with ethidium bromide, which is a subjective method requiring highly qualified staff. As a result, many attempts to design a colorimetric nonisotopic method for the detection of PCR products analogous to immunological reactions for enzyme immunoassay (EIA) have been attempted. Colorimetric reactions are much more sensitive, can be measured by simple photometers, and can be quantitative allowing more reliable and more objective interpretation of the results.

A notable difficulty with colorimetric approaches for detection is the unavailability of a specific method to capture the PCR products. There are three different currently available ways to capture PCR products: (1) hybridization with a probe attached to a solid-phase (microtiter well), (2) antibodies specific to RNA-DNA hybrids, which can be prepared to specifically capture hybrids formed between amplified DNA and specific RNA probes, and (3) specific labeling of the PCR products (usually biotinylation) by using special labeled primers, or nucleotides. Only hybridization with a probe provides sequence specific capture of the PCR fragments. However, the main disadvantage of hybridization is low efficiency of the process because of high dependence on DNA denaturation conditions. At annealing temperatures or at neutralization conditions after alkali denaturation, DNA forms a double-stranded structure. If the double-stranded DNA is denatured it can hybridize with an oligonucleotide probe and the product can be captured and detected; however, if the DNA is not denatured it cannot be captured, because there is no way for the probe to hybridize with the DNA at annealing conditions. Thus, the usual hybridization techniques are inefficient, since three different competing reactions occur simultaneously when standard annealing conditions are used: (1) probe binding, (2) restoration of the double-stranded form of the PCR fragments, and (3) nonspecific burial of the interacting region of the amplified DNA product inside of the macrostructure organized in the DNA.

To overcome two of the major challenges in PCR detection: a) the quantitative data challenges and b) new detection method, there has recently been developed a new method for real time detection of the PCR product. The new fluorescent assay system are based on the 5′ exonuclease activity of Taq DNA polymerase has been developed for detecting correctly amplified targets produced during the polymerase chain reaction (PCR). The method uses an oligonucleotide probe complementary to an internal region of the target sequence and included into each PCR reaction. The probe contains a fluorescent dye and a quencher. During the extension phase of PCR, Taq polymerase releases the dye from the quencher, thus increasing fluorescent yield of the dye. The assay is at least as sensitive as ethidium bromide staining, and eliminates the need for analysis of PCR products by gel electrophoresis. Completed PCR reactions are read in a luminescence spectrometer equipped with a microwell plate reader. Data is collected automatically and transferred to a spreadsheet.

Recently the rolling circle amplification technology is becoming a strong alternative to PCR for applications involving the detection of specific nucleic acid sequences. The method involves amplifying a circular nucleic acid probe produced following interaction of a nucleic acid probe with a target sequence whereby the circular nucleic acid probe is enriched prior to amplification. Enrichment reduces the level of background amplification by removing any linear nucleic acid probes, and may be enzymatic or non-enzymatic. Amplification may be by rolling circle amplification. The probe may be a padlock probe. The terminal sequences of the probe may form non-contiguous duplexes with the probe circularized through ligation of a capture ligand or spacer nucleic acid molecule between the two terminal sequences. The capture ligand or spacer nucleic acid molecule may be labeled, such as with biotin.

In summery both relative well characterized method such as PCR and more newly developed methods such as RCA all still requires modification of the biological material before detection still requires relative expensive highly specialized equipment not available in a typical medical doctor practice or big-clinical laboratory for routine molecular diagnostics blood testing.

The present invention provides novel compositions and methods which are utilized in a wide variety of nucleic acid-based procedures, and further provides other, related advantages.

### SUMMARY OF THE INVENTION

The invention includes devices to determine the presence or absence of a target analyte comprising a microsensor wherein the microsensor has a surface capable of binding to a target analyte. Upon binding of the analyte, the surface of the microsensor may undergo stress which results in deflection of the microsensor. In one embodiment the microsensor is in mechanical communication with a piezoelectric element and deflection is detected by a change in an electrical parameter of the piezoelectric element selected from the group consisting of voltage, resistance and current. In an alternate embodiment, deflection can be detected by use of a radiation source, such as a laser, to measure the deflection angle of the microsensor. In some embodiments, the binding of analyte to the surface results in an increase in mass which results in a gravitational deflection of the microsensor which is also detected by the piezoelectric element by way of one or more of its electrical properties. When a mass change of the microsensor is desired, it is preferred that the surface area of the microsensor capable of binding to the target analyte be maximized so as to enhance the microsensor deflection and hence the signal from the piezoelectric element.

In another aspect, the invention further includes an oscillator, an oscillator controller and a measuring device for measuring oscillation amplitude or resonance frequency of the microsensor. In one embodiment, the amplitude and/or resonant frequency of the microsensor is measured using a piezoelectric element or a laser source. In such embodiments, the oscillator is in direct mechanical communication or indirect mechanical communication (e.g., via a solid and/or fluid media) with the microsensor. The oscillator controller controls the oscillator so as to control the vibration frequency and/or amplitude of the microsensor.

When used to detect a change in mass of the microsensor, the oscillator controller is set to oscillate the microsensor at or near the resonance frequency of the microsensor so as to establish a baseline. Thereafter, a target analyte is contacted with the microsensor and the amplitude or resonance frequency of the microsensor is determined by measuring (1) one or more parameters of the piezoelectric element or (2) the reflective output of a surface of the microsensor when a laser source is used. Binding of a target analyte to the microsensor results in a change in the amplitude and/or resonance frequency of the microsensor.

In another aspect, the invention includes utilizing the aforementioned device in a method wherein both microsensor deflection and a change in microsensor resonance amplification and/or resonance frequency is measured. Such measurements may be repeated to measure the kinetics of a binding reaction between target analyte and the surface of the microsensor.

The microsensors used in the aforementioned devices include micro-cantilevers and micromembranes, each of which are well known in the art. In general, microsensors are preferably used in pairs. One microsensor is treated with an agent which specifically binds to a target analyte and is the measuring microdevice whereas another microdevice is not so treated and is referred to as a reference microdevice. The reference microdevice is used as need be to correct for non-specific environmental factors such as mass flow, temperature and the like.

One or more of the aforementioned devices can be incorporated into a microfluidic device. In such embodiments, at least one device is positioned in a microfluidic channel or chamber wherein fluid flows past the surface of the microsensor. A multiplicity of such microsensors each having different analyte specificity can be incorporated into the channel and/or chamber for multiplex analyte analysis of a test sample.

Another aspect the invention is directed to a method for determining the presence or absence of a target analyte, such as a nucleic acid, in a test sample. In the case of nucleic acids, in one embodiment, the method comprises contacting the target nucleic acid with a piezoelectric biosensor comprising a microsensor having a surface which comprises an immobilized probe nucleic acid which hybridizes to a first region of the test nucleic acid. When so bound, a hybridization complex is formed. The formation of this complex and therefore the presence of the target analyte can be detected by measuring the deflection of the microsensor and/or a change in the resonance amplitude or resonance frequency of the microsensor.

In the hybridization complex formed by the immobilized probe and test nucleic acid, the first region of the target nucleic acid is hybridized to the immobilized probe and forms a double-stranded region. A second region of the target nucleic acid, adjacent to the first region, is single-stranded in the hybridization complex. The hybridization complex is then exposed to a condition (e.g., nucleotide extension via a polymerase or oligonucleotide ligation via a ligase) which results in the extension of the probe nucleic acid in the hybridization complex using the second region in the test nucleic acid region as template. Thereafter a parameter of the piezoelectric element or a laser is used to provide an indication of whether or not the probe nucleic acid has been extended.

In a further aspect, the probe nucleic acid comprises a terminal end region comprising the last 3 nucleotides and preferably the a terminal nucleotide which in one embodiment contains one or more base pair matches or mismatches with the opposing nucleotide(s) in the first region of the test nucleic acid in the hybridization complex. In another embodiment, the base pair matching or mismatching occurs in the second region of the test nucleic acid. For example, in the case of oligonucleotide hybridization to the second region and subsequent ligation (IEOLA), base pair matches or mismatches may be in the end region of the probe or the end region of the oligonucleotide adjacent to the immobilized probe. In either case, it is preferred that the match or mismatch occur at the terminal nucleotide portion. Extension of the immobilized primer provides an indication of the sequence present in the target nucleic acid complementary to said end regions.

Alternatively, the piezoelectric element need not be present and probe extension can be detected by measuring deflection angle using a radiation source, e.g., a laser.

In a further aspect of the invention, the extension of the probe nucleic acid can be measured by oscillating the microsensor and detecting a change in amplitude and/or resonance frequency via (1) the resistence, current or voltage of the piezoelectric element and/or (2) use of a light source such as a laser.

In conjunction with detection of nucleic acids, optionally, an amplification reaction such as PCR or LCR may be performed prior to or simultaneously with the contacting of the target nucleic acid with the biosensor.

Other embodiments which provide sequence information include a polymerase based probe extension wherein the separate addition of one or more of the possible nucleotide triphosphates results in selective probe extension.

### BRIEF DESCRIPTION OF THE DRAWINGS

FIG. 1 is an image of cantilever-based sensor with piezoresisitive read-out. In the images of the two cantilevers are seen from the top. The shaded cantilever is gold-coated on the top side for thiol-modified DNA-oligo immobilization whereas the other cantilever serves as reference.

FIG. 2 is a schematic drawing of the cantilever system. The piezoresisitive read-out system consists of the-four resistors labelled 1, 2, 3 and-4. Resistors 2 and 3 are the test cantilever and the reference cantilever. Resistors 1 and 4 are internal support resistors.

FIG. 3 is a schematic drawing of the piezoresisitive read-out system. A voltage V=2 V is applied across the Wheatstone bridge and a change in the resistance of the piezoresistor on the measurement cantilever, induced by a change in the surface stress of the cantilever, results in a change in the output voltage VO from the Wheatstone bridge

FIG. 4 is a schematic drawing of the main experimental set-up. The channel consists of a total of 2 cantilever systems in a closed micro system. Either the probes or the reaction mixture can enter the system at inlet 1 and inlet 2.

FIG. 5A is an image of a 2×5-channel cantilever. The inlets are at 1 and 2. The outlets are 3 and 4; 5 is one of the 10 cantilever channels. FIG. 5B is the actual size of micro-cantilever FIG. 5A.

FIG. 6 depicts the steps in the immobilization of probe on a gold coated micrometer sized cantilever sensor. The drawing shows a cut perpendicular to the surface through the sensor. In FIG. 6A, the gold surface is cleaned in situ by a controlled AR etch. In FIG. 6B, the sensor is exposed to a 10 μM concentration of a thiol-modified DNA. The probe binds to the gold surface via the thiol-group in the 3′ end of the probe.

FIG. 7 demonstrates the surface stress change, Δσ, as a function of time for a cantilever sensor exposed to thiol-modified DNA probes (wCF probe). At time 10 second the sensor is exposed to the probe. The sensor reacts immediately to the probe, starting by a short release of surface stress. Thereafter, the surface stress increases to its saturation value within 100 seconds. The immobilization curve is fitted by two Langmuir isotherms and the Langmuir model describes well the entire stress curve.

FIG. 8 shows a three dimensional view of the cantilever having one test cantilever and one reference cantilever. The cantilever unit are places on a flat surface where the temperature can be controlled. The flat surface is part of a thermo-cycler in PCR amplification for example.

FIG. 9 is a cross-section illustration of the micro-cantilever unit. Two cantilevers are shown.

FIG. 10 is a flowchart for mutation/SNP detection

FIG. 11-13 described the principle for measure a SNP. The three figure illustrate three situations: 1) no SNP in the DNA. 2) Homozygous mutation in the DNA. 3) Heterozygous mutation in the DNA. A typical hybridization assay protocol for detecting a target nucleic acid in a complex population of nucleic acids is described as follows: A probe containing a nucleotide complementary to the SNP position of the target at the very 3 prime end is immobilized on one micro-cantilever at its 5′ end (probe 1). Within the surroundings of the first micro-cantilever a second micro-cantilever are immobilized with a probe having the wild type sequence (probe 2). Two primers are designed for PCR amplification of a PCR product containing the potential SNP site. Normally the probe sites are located close to one of the primer sites. The following events may occur simultaneously in the closed interaction chamber: 1 ) DNA amplification of target nucleic acid molecule in solution using the two above primers 2) hybridization of amplified target nucleic acid molecule to the probe 1 and probe 2 immobilized on two different cantilevers. The target nucleic acid molecule hybridizes to the 3′ region of the immobilized probe sequence, to form a hybridization complex that has a 3′ terminus; 3) 3′ extension of the DNA strand hybridized to the immobilized probe on the surface of the cantilever. A) If the DNA tested has the SNP site, probe 1 will hybridize more efficiently to the DNA compared to probe 2 where a 3′ mismatch will inhibit the 3′ extension reaction of the DNA strand hybridized to the immobilized probe on the surface of the cantilever.

FIG. 14 is a schematic representation of the DNA fragment containing the Δ508 mutation in the cystic fibrosis gene. 1, 2) Primer for the PCR amplification of a 419 bp DNA fragment. 3) the DNA fragment complementary to the Δ508 probe, 4) the PCR product.

FIG. 15 is a flowchart for specific detection of RNA molecules

FIG. 16 shows the principle for measuring an RNA or DNA molecule. 1)The surface of the cantilever; 2) the probe, 4, 5) the primers for PCR amplification, 6) the DNA fragment complementary to the probe, 7) the PCR fragment. A typical hybridization assay protocol for detecting a target nucleic acid in a complex population of nucleic acids is described as follows: 16A) A probe containing a sequence complementary to the target RNA/DNA molecule are immobilized on one micro-cantilever. Two primer are design for PCR amplification of a PCR product containing the probe site. Normally the probe site is located close to one of the primer sites. The following events may occur simultaneously in the closed chamber of the device: 1) DNA amplification of target nucleic acid molecule in solution using the two above primers 2) hybridization of amplified target nucleic acid molecule to the probe immobilized on the surface of the cantilever, to thereby form a hybridization complex. 16B) If the DNA or RNA are present in the complex population of nucleic acids tested, the probe will hybridize to the DNA and the 3′ extension reaction will take place. The mass increase as a result of primer extension can be directly observed due to different mechanical stress detection levels.

FIG. 17 is a schematic representation of the Interleukin 6 DNA fragment. 1, 2) Primer for the PCR amplification of a 628 bp DNA fragment. 3) the DNA fragment complementary to the IL 6 probe, 4) the PCR product.

FIG. 18 is a schematic presentation of the GAPD DNA fragment. 1, 2) Primer for the PCR amplification of a 160 bp DNA fragment. 3) the DNA fragment complementary to the GAPD probe, 4) the PCR product.

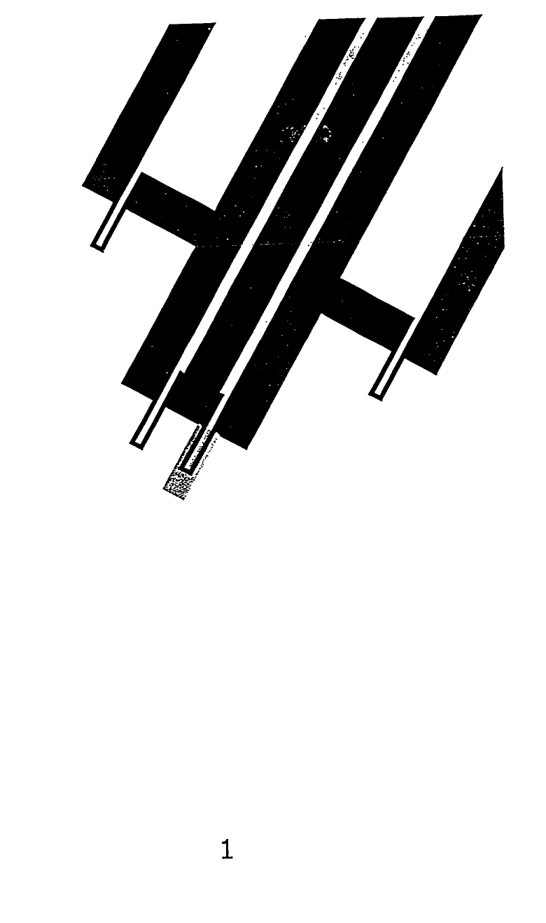

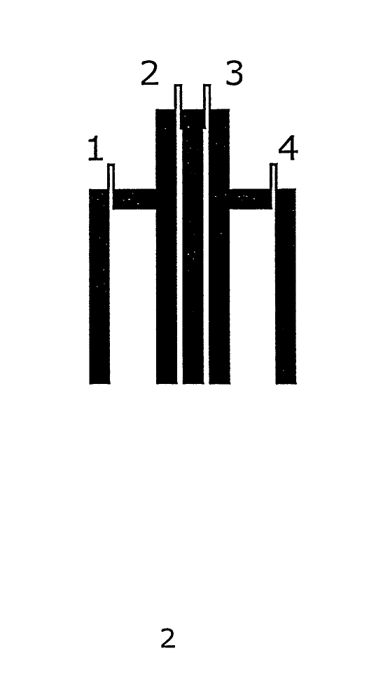

### DETAILED DESCRIPTION OF THE INVENTION

The present invention provides a microsensor device and method for the detection of target analytes. In addition the invention provides a multi-component device for the simultaneous detection of multiple analytes of interest. The microsensor device may include multiple chambers for independent measurement or detection of target analytes.

The present invention is directed to a device that relies on microsensor such as micro-cantilevers or micro-membranes for detection of target analytes. In a preferred embodiment the microdevice is a micro-cantilever. By “micro-cantilevers” or “cantilevers” or grammatical equivalents herein is meant devices in which changes in the mechanical properties of the micro-cantilever are used to detect changes in the environment of the micro-cantilever. The micro-cantilever is typically of the order of 100 microns long, 10 microns wide and one micron thick. The micro-cantilevers are made of a material such as silicon, silicon nitride, glass, metal or combinations of any of these, using micro-machining techniques.

By “micro-membrane” is meant a thin disk preferably pre-coated with a wide range of films selected from metals, polymers, ceramics to bio-molecules. The micro-membrane may be oscillated at its resonance frequency. A large number of different micromembranes exist, see for example E. Quandt, K. Seemann, Magnetostrictive Thin Film Microflow Devices, Micro System Technologies 96, pp. 451-456, VDE-Verlag GmbH, 1996, which is expressly incorporated herein by reference.

A change in a mechanical property of a microsensor can, for example, be stress formation in the microsensor due to changes in surface tension of the microsensor. Stress formation can also occur due to changes in temperature of the microsensor due to a bimorph effect, if the microsensor is made of two materials with different thermal expansion coefficients. Such stress often results in the deflection or bending of the microsensor. Stress can also be the result of an increase or decrease on the mass of the microsensor which can result in deflection of the microsensor. Such stress or deflection in the micro-cantilever can be detected in a variety of ways. If deflection of the microsensor occurs, the deflection can be detected for example by a laser beam, a reflecting surface of the micro-cantilever and an optical detector to measure the deflection angle.

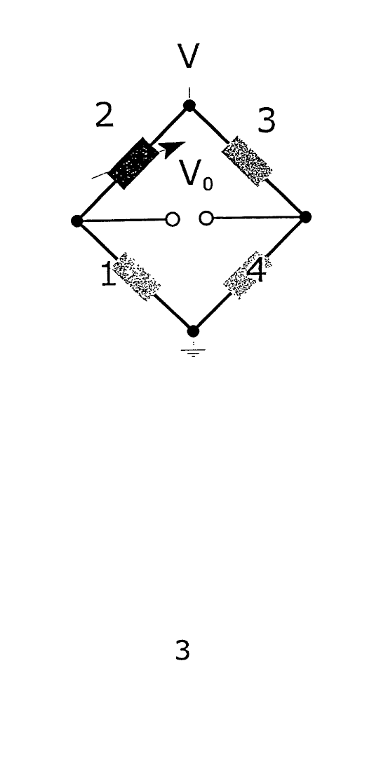

An alternative and preferred method of detecting changes on a microsensor is detection of changes in an electrical property of a piezoelectric element integrated with the microsensor. This method has an advantage in that it does not require optical access to the microsensor. In general, at least one electrical parameter of the piezoelectric element is measured to detect a change in the microsensor. Such parameters include resistance, current or voltage. Placing the piezoelectric element within an arm of a Wheatstone Bridge such as set forth in FIG. 3, provides a means to detect one or more electrical parameters of the piezoelectric element.

Additionally or alternatively, changes in resonance frequency or amplitude can be used to detect a change in a mechanical property of the microsensor.

A change of mass of the microsensor occurs when sufficient material binds to the micro-sensor, so as to produce a change in the resonance frequency or amplitude of vibration of the microsensor. Such changes can be monitored by use of an oscillator to vibrate the microsensor at or near a frequency near its resonance frequency. Changes in the amplitude or resonant frequency of the dynamic bending of the microsensor can be measured using the piezoelectric element and measuring one or more electrical parameters. Alternatively, a laser or other source of radiation may be used to detect the sequence frequency and/or amplification of vibration of the microsensor.

Recently, acoustic network analysis (Su, H. & Thompson, M Biosensors & Bioelectronics. 10, 329-340, 1995), and quartz balance resonators; Caruso, F., Furlong, D. N., Nilkura, K. & Okahata, Y. Colloids and Surfaces B. 10,199-204, 1998), has been used to investigate the kinetics of DNA sensor layer formation. Moreover, Scanning Tunnelling Microscopy (Zhao, Y. et al. Analytica Chimica Acta. 388, 93-101, 1999), and Atomic Force Microscopy (Hegner, M., Dreier, M., Wagner, P., Semenza, G. & Guntherodt, H. J. J. Vac. Sci. Technol. B.14,1418-1421, 1996)), have been used to study local mechanical and chemical properties of immobilized DNA. For stress formation studies in ambient and aqueous environments, micrometer-sized cantilevers with optical read-out have proven very sensitive (Berger, R., Gerber, Ch., Lang, H. P. & Gimzewski, J. K. Microelectronic Engineering. 35, 373-379,1997) and O'Shea, S. J., Welland, M. E. J. Vac. Sci. Technol. B. 14, 1383-1385, 1996). Other sensors utilizing cantilevers are described in U.S. Pat. Nos. 5,552,724, 4,847,193, 5,445,008, 5,719,324, 6,096,559, 5,739,425 and 5,807,758, all of which are expressly incorporated by reference herein. The invention will be further described within the context of the microsensor being a cantilever. It is to be understood, however, that other microsensors such as micromembranes may be substituted for such micro-cantilevers.

Basically, a biochemical reaction at the cantilever surface can be monitored as a bending of the cantilever due to a change in the surface stress (N/m) on one side of the cantilever relative to the other. Surface stress changes in self-assembled alkanethiols on gold have earlier been measured in air by this technique (Berger, R. et al. Science. 276, 2021-2024,1997), and surface stress changes of approximately 10⁻⁵ N/m can be resolved by cantilever-based methods (Berger, R., Gerber, Ch., Lang, H. P. & Gimzewski, J. K. Microelectronic Engineering. 35, 373-379 (1997)). Recently J. Friz et. al. Science. 288, 316-318, 2000) showed the used of cantilever for DNA-oligo hybridization using a optical readout system. There have previously been developed cantilever-based sensors with integrated piezoresistive read-out (Thaysen, J., Boisen, A., Hansen, O. & Bouwstra, S. Proceedings of Transducers 99,1852-1855, Sendai 1999). Other methods of detecting utilizing cantilevers are described in U.S. Pat. Nos. 6,203,983, 5,658,732, 5,763,768, 5,972,617, 5,345,815, 5,445,008, 5,719,324 and 6,096,559, all of which are expressly incorporated herein by reference.

Until now stress changes on cantilever sensors have been registered by monitoring the bending of the cantilever using optical leverage. However, integrated readout greatly facilitates operation in solutions since the refractive indices of the liquids do not influence the detection (Raiteri, R., Nelles, G., Butt, H. -J., Knoll, W. & Skladal, P. Sensors and Actuators B, 61, 213-217, 1999). Moreover, the piezoresistive cantilever yields a direct measure of the surface stress, thus eliminating the discussion of cantilever bending. Each sensor has a built-in reference cantilever, which makes it possible to subtract background drift directly in the measurement. The two cantilevers are connected in a Wheatstone bridge and the deflection of the measurement cantilever is detected as the output voltage from the Wheatstone bridge. The sensors have been used in liquid experiments (Boisen A., Thaysen J., Jensenius H., & Hansen, O. Ultramicroscopy, 82,11-16, 2000), where the reference cantilever is seen to be important for minimizing thermal drift and noise due to liquid flow. The noise level of the sensor is reduced by a factor of 25 when applying the reference cantilever and the Wheatstone bridge configuration. Accordingly, in a preferred embodiment, the sensor of the invention includes a reference cantilever and a measuring cantilever. By “measuring cantilever” is meant the cantilever to which the binding ligand (that binds the target analyte) is capable of binding. A “reference cantilever” is a cantilever which does not specifically bind target analyte.

Based on the dimensions of the cantilever and the gauge factor of the silicon piezoresistor the output voltage from the Wheatstone bridge can be transformed directly to a measure of surface stress. An increase in the output voltage corresponds to a compressive stress in the formed layer, whereas a decreasing signal is a result of a tensile stress.

Accordingly, the sensor of the invention includes at least one micro-cantilever and a detector to detect a change in a property of the micro-cantilever.

The apparatus of the invention also includes a plurality of cantilevers for the detection of a plurality of target analytes. Preferably, the cantilevers of the invention are positioned in a channel or chamber. The channel or chamber has inlet or outlet ports which allow for the introduction of samples into the channel or chamber for analysis of target samples. In one embodiment, the sample may be separated, for example, into different channels or chambers for separate analysis. That is, in one embodiment multiple samples can be analyzed simultaneously. In an alternative embodiment multiple target analytes can be analyzed from a single sample. That is, a plurality of discrete microsensors may be contained within a single chamber. In this embodiment the individual microsensors may be used to detect discrete target analytes from a single sample.

Accordingly, the micro-cantilever based device of the invention is used to detect target analytes in samples. By “target analyte” or “analyte” or grammatical equivalents herein is meant any molecule, compound or particle to be detected. As outlined below, target analytes preferably bind to binding ligands, as is more fully described herein. Preferably the binding ligands are immobilized to a surface of the micro-cantilever. As will be appreciated by those in the art, a large number of analytes may be detected using the present methods; basically, any target analyte, for which a binding ligand exists, may be detected using the methods and apparatus of the invention.

Suitable analytes include organic and inorganic molecules, including biomolecules. In a preferred embodiment, the analyte may be an environmental pollutant (including heavy metals, pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc)(detection of antigen antibody interactions are described in U.S. Pat. Nos. 4,236,893, 4,242,096, and 4,314,821, all of which are expressly incorporated herein by reference); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

In a preferred embodiment, the target analyte and binding ligands are nucleic acids. By “nucleic acid” or “oligonucleotide” or grammatical equivalents herein means at least two nucleotides covalently linked together.

In a preferred embodiment, the present invention provides methods of detecting target nucleic acids. By “target nucleic acid” or “target sequence” or grammatical equivalents herein means a nucleic acid sequence on a single strand of nucleic acid. The target sequence may be a portion of a gene, a regulatory sequence, genomic DNA, cDNA, RNA including mRNA and rRNA, or others. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. Target sequences also include the result or product of an amplification reaction.

A nucleic acid of the present invention will generally contain phosphodiester bonds, although in some cases, as outlined below, nucleic acid analogs that may have alternate backbones may be used. Preferably, the nucleic acid target analyte is a polynucleotide. Nucleic acid analogs are preferably used, if at all, as immobilized probes (binding ligand) on the surface of a microsensor. Such nucleic acid analytes have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). In addition, locked nucleic acids (LNA) find use in the invention. LNA are described in more detail in Wengel el al.; J. Org Chem 63; 10035-9 1998, which is expressly incorporated herein by reference. Several nucleic acid analogs are described in Rawls, C & E News Jun. 2, 1997 page 35. All of these references are hereby expressly incorporated by reference. These modifications of the ribose-phosphate backbone may be done to facilitate the addition of labels or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occuring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration.

The nucleic acids whether a target nucleic acid, probe or elongation product, for example of a polymerase or a ligase, may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides and nucleoside and nucleotide analogs, and modified nucleosides such as amino modified nucleosides. In addition, “nucleoside” includes non-naturally occuring analog structures. Thus for example the individual units of a peptide nucleic acid, each containing a base, are referred to herein as a nucleoside.

As is outlined more fully below, probes (including amplification primers) are made to hybridize to target sequences to determine the presence or absence of the target sequence in a sample. Generally speaking, this term will be understood by those skilled in the art.

The target sequence may also be comprised of different target domains, for example, in “sandwich” type assays as outlined below, a first target domain of the sample target sequence may hybridize to an immobilized probe or primer on a microsensor, i.e. cantilever, and a second target domain may hybridize to a solution-phase probe or primer. In addition, the target domains may be adjacent (i.e. contiguous) or separated. For example, when ligation techniques are used, a first primer may hybridize to a first target domain and a second primer may hybridize to a second target domain; either the domains are adjacent, or they may be separated by one or more nucleotides, coupled with the use of a polymerase and dNTPs, as is more fully outlined below. In such cases, at least one of the primers is immobilized on the surface of a microsensor and a ligase is used to covalently join the probe.

In another preferred embodiment, the target analyte is a protein. As will be appreciated by those in the art, there are a large number of possible proteinaceous target analytes that may be detected using the present invention. By “proteins” or grammatical equivalents herein is meant proteins, oligopeptides and peptides, derivatives and analogs, including proteins containing non-naturally occurring amino acids and amino acid analogs, and peptidomimetic structures. As discussed below, when the protein is used as a binding ligand, it may be desirable to utilize protein analogs to retard degradation by sample contaminants.

These target analytes may be present in any number of different sample types, including, but not limited to, bodily fluids including blood, lymph, saliva, vaginal and anal secretions, urine, feces, perspiration and tears, and solid tissues, including liver, spleen, bone marrow, lung, muscle, brain, etc.

Accordingly, the present invention provides a single or multi-component devices for the detection of target analytes. As noted above, the device includes a detection channel or chamber that includes at least one cantilever and may preferably contain at least 4, 5, 10, 20, 30, 40, 50 or 100 cantilevers. In a preferred embodiment the chamber includes at least 100 cantilevers. As described herein, the cantilevers are coupled to a detector.

In one embodiment the device includes a single channel or chamber for the amplification and detection of target nucleic acids. Alternatively, the device may comprise more than one channel or chamber; for example, there may be a “sample treatment” or “sample preparation” channels or chambers that interfaces with a separate “detection” channel or chamber. By “channel” is meant a path or trough through which a sample flows, generally between chambers, although in some embodiments reactions can occur in the channels themselves. By “chamber” is meant a closed or closeable portion of the microfluidic device in which samples are manipulated and/or detected. While much of the discussion below emphasizes reactions occurring in chambers, it is appreciated that any of the reactions or manipulations also can occur in channels.

Generally, when nucleic acids are to be detected and nucleic acids serve as the probes or primers, two general schemes find use in the invention. In one embodiment the target analyte is amplified to produce amplicons. Amplicons are then detected with the microsensor. In another embodiment, the target analyte hybridizes with the probe or primer immobilized on the microsensor. The probe or primer is modified and the modification, which generally includes a change in the mass of the probe or primer, is detected. As one of skill in the art appreciates, “target analytes” can include both targets from samples or products of an amplification reaction, i.e. amplicons. That is, amplicons can serve as target analytes. The immobilized probe can then be modified as a result of hybridization with the amplicons.

As noted previously, detection of target analytes can occur by hybridization of a target to a probe immobilized on the surface of a substrate. Detection also can occur by detecting a modification of the immobilized probe or primer. This results in the formation of a “modified primer”. While there are a variety of types of modifications, generally modifications that find use in the present invention are those that result in a change in mass of the immobilized probe or primer. That is, in general the probe or primer will be modified by extension such as by a DNA polymerase or ligase. Sandwich assays also find use in detection of target analytes.

As discussed herein, it should be noted that the sandwich assays can be used for the detection of primary target sequences (e.g. from a patient sample), or as a method to detect the product of an amplification reaction as outlined above; thus for example, any of the newly synthesized strands outlined above, for example using PCR, LCR, NASBA, SDA, etc., may be used as the “target sequence” in a sandwich assay. Sandwich assays are described in U.S. Ser. No. 60/073,011 and in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference. In addition these target sequences can be used as templates for other assays that result in modification of the immobilized primers.

Single Base Extension (SBE) is an extension assay that results in the incorporation of a nucleotide into a primer sequence when the primer sequence is complementary to or hybridized with a target sequence. The nucleotide incorporated into the primer is complementary to the nucleotide at the corresponding position of the target nucleic acid. Accordingly, the immobilized primer is extended, i.e. modified, and is detected by the device of the invention. As such, detection of a change in the immobilized primer is an indication of the presence of the target analyte.

Oligonucleotide-ligation assay is an extension of PCR-based screening that uses an ELISA-based assay (OLA, Nickerson et al., Proc. Natl. Acad. Sci. USA 87:8923, 1990) to detect the PCR products that contain the target sequence. Briefly, the OLA employs two adjacent oligonucleotides: a “reporter” probe and an “anchor” probe. The two oligonucleotides are annealed to target DNA and, if there is perfect complementarity, the two probes are ligated by a DNA ligase. The ligated probe is then captured by the probe on the cantilever.

Alternatively, one of the OLA primers is immobilized on the microsensor. Upon ligation, the mass on the microsensor is increased. The mass increase is detected as an indication of the presence of the target analyte.

In this and other embodiments, a heating and/or cooling module may be used, that is either part of the reaction chamber or separate but can be brought into spatial proximity to the reaction module. Suitable heating modules are described in U.S. Pat. Nos. 5,498,392 and 5,587,128, and WO 97/16561, incorporated by reference, and may comprise electrical resistance heaters, pulsed lasers or other sources of electromagnetic energy directed to the reaction chamber. It should also be noted that when heating elements are used, it may be desirable to have the reaction chamber be relatively shallow, to facilitate heat transfer; see U.S. Pat. No. 5,587,128.

In one embodiment, the devices of the invention includes a separate detection module. That is, when the reaction channel or chamber does not include the microsensors, a separate detection channel or chamber is needed. It should be noted that the following discussion of detection modules is applicable to the microsensor when the microsensors are found in the reaction channel or chamber.

Accordingly, the present invention is directed to methods and compositions useful in the detection of biological target analyte species such as nucleic acids and proteins. In general, the detection module is based on binding partners or bioactive agents attached to microsensors as described herein.

That is, each microsensor comprises a bioactive agent. By “candidate bioactive agent” or “bioactive agent” or “chemical functionality” or “binding ligand” herein is meant any molecule, e.g., protein, oligopeptide, small organic molecule, coordination complex, polysaccharide, polynucleotide, etc. which can be attached to a microsensor. Preferred bioactive agents include biomolecules including peptides, nucleic acids, saccharides, fatty acids, steroids, purines, pyrimidines, derivatives, structural analogs or combinations thereof. Particularly preferred are nucleic acids and proteins.

In one preferred embodiment, the bioactive agents are naturally occurring proteins or fragments of naturally occurring proteins. Thus, for example, cellular extracts containing proteins, or random or directed digests of proteinaceous cellular extracts, may be used. In this way libraries of procaryotic and eukaryotic proteins may be made for screening in the systems described herein. Particularly preferred in this embodiment are libraries of bacterial, fungal, viral, and mammalian proteins, with the latter being preferred, and human proteins being especially preferred.

In a preferred embodiment, the bioactive agents are peptides of from about 5 to about 30 amino acids, with from about 5 to about 20 amino acids being preferred, and from about 7 to about 15 being particularly preferred.

In a preferred embodiment, the bioactive agents are nucleic acids as defined above (generally called “probe nucleic acids”, “primers” or “candidate probes” herein). As described above generally for proteins, nucleic acid bioactive agents may be naturally occurring nucleic acids, random nucleic acids, or “biased” random nucleic acids. For example, digests of procaryotic or eukaryotic genomes may be used as is outlined above for proteins.

When the bioactive agents are nucleic acids, they are designed to be substantially complementary to target sequences. As noted above, the term ‘target sequence” or grammatical equivalents herein means a nucleic acid sequence on a single strand of nucleic acid.

A probe nucleic acid (also referred to herein as a primer nucleic acid) is then contacted to the target sequence to form a hybridization complex. Generally, the probe nucleic acid is immobilized on the surface of a microsensor or micro-cantilever. By “primer nucleic acid” herein is meant a probe nucleic acid that will hybridize to some portion, i.e. a domain, of the target sequence. Probes of the present invention are designed to be complementary to a target sequence (either the target sequence of the sample or to other probe sequences, as is described below), such that hybridization of the target sequence and the probes of the present invention occurs. As outlined below, this complementarity need not be perfect; there may be any number of base pair mismatches which will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under normal reaction conditions.

A variety of hybridization conditions may be used in the present invention, including high, moderate and low stringency conditions; see for example Maniatis et al., Molecular Cloning: A Laboratory Manual, 2d Edition, 1989, and Short Protocols in Molecular Biology, ed. Ausubel, et al, hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 sodium ion, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide. The hybridization conditions may also vary when a non-ionic backbone, i.e. PNA is used, as is known in the art. In addition, cross-linking agents may be added after target binding to cross-link, i.e. covalently attach, the two strands of the hybridization complex.

Thus, the assays are generally run under stringency conditions which allows formation of the hybridization complex only in the presence of target. Stringency can be controlled by altering a step parameter that is a thermodynamic variable, including, but not limited to, temperature, formamide concentration, salt concentration, chaotropic salt concentration pH, organic solvent concentration, etc.

These parameters may also be used to control non-specific binding, as is generally outlined in U.S. Pat. No. 5,681,697. Thus it may be desirable to perform certain steps at higher stringency conditions to reduce non-specific binding.

The size of the probe or primer nucleic acid may vary, as will be appreciated by those in the art, in general varying from 5 to 500 nucleotides in length, with primers of between 10 and 100 being preferred, between 15 and 50 being particularly preferred, and from 10 to 35 being especially preferred, depending on what is required for detection and/or amplification as is discussed below.

In a preferred embodiment, each microsensor comprises a single type of bioactive agent, although a plurality of individual bioactive agents are preferably attached to each microsensor, as described herein. In addition, as described above, the microsensor is in communication with a detector such that the presence of the target analyte can be determined.

In a preferred embodiment, the devices of the invention include a reaction module. This can include either physical, chemical or biological alteration of one or more sample components. Alternatively, it may include a reaction module wherein the target analyte alters a second moiety that can then be detected; for example, if the target analyte is an enzyme, the reaction chamber may comprise a substrate that upon modification by the target analyte, can then be detected by binding to a microsensor. In this embodiment, the reaction module may contain the necessary reagents, or they may be stored in a storage module and pumped as outlined herein to the reaction module as needed.

Alternatively, the target analyte serves as a substrate for an enzymatic reaction such as a polymerase or ligase extension reaction, but the target itself is not altered or consumed. Rather, the immobilized probe or primer in the microsensor is modified in a template or target analyte dependent manner.

In a preferred embodiment, the reaction module includes a chamber for the chemical modification of all or part of the sample before or during analyte detection. That is, in one embodiment there is a separate reaction module and a separate detection module. In an alternative embodiment the reaction occurs in the detection module. This allows for simultaneous modification and detection of analytes.

Chemical modifications include, but are not limited to chemical cleavage of sample components (CNBr cleavage of proteins, etc.) or chemical cross-linking. PCT US97/07880, hereby incorporated by reference, lists a large number of possible chemical reactions that can be performed in the devices of the invention, including amide formation, acylation, alkylation, reductive amination, Mitsunobu, Diels Alder and Mannich reactions, Suzuki and Stille coupling, etc. Similarly, U.S. Pat. Nos. 5,616,464 and 5,767,259 describe a variation of ligation chain reaction (LCR; sometimes also referred to as oligonucleotide ligation amplification or OLA) that utilizes a “chemical ligation” of sorts.

In a preferred embodiment, the reaction module includes a chamber for the biological alteration of all or part of the sample before or during analyte detection. For example, enzymatic processes including nucleic acid amplification and other nucleic acid modifications including ligation, cleavage, circularization, supercoiling, methylation, acetylation; hydrolysis of sample components or the hydrolysis of substrates by a target enzyme, the addition or removal of detectable labels, the addition or removal of phosphate groups, protein modification (acylation, glycosylation, addition of lipids, carbohydrates, etc.), the synthesis/modification of small molecules, etc.

Alternatively, the modification or alteration may occur in the immobilized primer as a result of hybridization with the target molecule.

In a preferred embodiment, the target analyte is a nucleic acid and the biological reaction chamber allows amplification of the target nucleic acid. Suitable amplification techniques include polymerase chain reaction (PCR), reverse transcriptse PCR (RT-PCR), ligase chain reaction (LCR), and Invader™ technology. Techniques utilizing these methods are well known in the art. In this embodiment, the reaction reagents generally comprise at least one enzyme (generally polymerase), primers, and nucleoside triphosphates as needed. As described herein, the amplification reactions can occur in a chamber or channel separate from the detection chamber. Alternatively, the amplification can occur in the detection chamber. As amplification proceeds, the amplicons hybridize to the immobilized probe on the microsensor in the detection chamber resulting in a detectable change in a property of the microsensor as outlined herein.

Alternatively, the amplicons serve as templates for subsequent reactions that result in a modification of the immobilized primer. Such modifications are discussed more fully below and include primer extension that results in lengthening the primer. Also, the primer can be ligated to another probe or primer such that the immobilized primer is lengthened.

General techniques for nucleic acid amplification are discussed below. In most cases, double stranded target nucleic acids are denatured to render them single stranded so as to permit hybridization of the primers and other probes of the invention. A preferred embodiment utilizes a thermal step, generally by raising the temperature of the reaction to about 95° C., although pH changes and other techniques such as the use of extra probes or nucleic acid binding proteins may also be used. In one embodiment isothermal amplification is preferred.

In addition, the different amplification techniques may have further requirements of the primers, as is more fully described below.

Once the hybridization complex between the primer and the target sequence has been formed, an enzyme, sometimes termed an “amplification enzyme”, is used to modify the immobilized primer. As for all the methods outlined herein, the enzymes may be added at any point during the assay, either prior to, during, or after the addition of the primers. The identification of the enzyme will depend on the amplification technique used, as is more fully outlined below. Similarly, the modification will depend on the amplification technique, as outlined below, although generally the first step of all the reactions herein is an extension of the primer, that is, nucleotides or oligonucleotides are added to the primer to extend its length.

In some embodiments, once the enzyme has modified the primer to form a modified primer, the hybridization complex is disassociated. By “modified primer” is meant a primer that has been changed or altered in a detectable manner. Generally a modified primer is lengthened by the addition of at least one nucleotide.

During amplification generally, the amplification steps are repeated for a period of time to allow a number of cycles, depending on the number of copies of the original target sequence and the sensitivity of detection, with cycles ranging from 1 to thousands, with from 10 to 100 cycles being preferred and from 20 to 50 cycles being especially preferred.

In one embodiment, after a suitable time or amplification, the amplicon is moved to a detection module and incorporated into a hybridization complex with a probe immobilized on the surface of a microsensor, as is more fully outlined below. The hybridization complex is attached to a microsensor and detected, as is described below.

In an alternative embodiment, amplification occurs in the detection chamber (described more fully below). That is, amplification and detection occur in the same chamber. In one embodiment amplification proceeds by using at least two solution phase primers. Following amplification, amplicons hybridize with probes or primers immobilized on the surface of the microsensor to form hybridization complexes. Upon hybridization with the immobilized probe, the presence of the target analyte is detected. In a preferred embodiment, the hybridization complex is used as a template for further reactions that result in the modification of the immobilized probe. Such reactions include extension reactions such as single base extension (SBE), template dependent nucleic acid synthesis or the oligonucleotide ligation assay (OLA) described in more detail herein.

In an alternative embodiment amplification and primer extension proceeds by the use of a solution-phase primer and a primer immobilized on the surface of the microsensor.

In yet another alternative embodiment, amplification proceeds by the use of primer pairs immobilized on the surface of a microsensor. That is, both amplification primers are immobilized on the surface of the microsensor. As such, upon amplification of the target analyte, the amplicons also are immobilized on the surface of the microsensor.

In a preferred embodiment, the amplification is target amplification. Target amplification involves the amplification (replication) of the target sequence to be detected, such that the number of copies of the target sequence is increased. Suitable target amplification techniques include, but are not limited to, the polymerase chain reaction (PCR), strand displacement amplification (SDA), and nucleic acid sequence based amplification (NASBA) and the ligase chain reaction (LCR).

In a preferred embodiment, the target amplification technique is PCR. The polymerase chain reaction (PCR) is widely used and described, and involves the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, “reverse transcriptase PCR” or “RT-PCR”, “biotin capture PCR”, “vectorette PCR”, “panhandle PCR”, and “PCR select cDNA subtration”, among others.

In general, PCR may be briefly described as follows. A double stranded target nucleic acid is denatured, generally by raising the temperature, and then cooled in the presence of an excess of a PCR primer, which then hybridizes to the first target strand. A DNA polymerase then acts to extend the primer, resulting in the synthesis of a new strand forming a hybridization complex. The sample is then heated again, to disassociate the hybridization complex, and the process is repeated. By using a second PCR primer for the complementary target strand, rapid and exponential amplification occurs. Thus PCR steps are denaturation, annealing and extension. The particulars of PCR are well known, and include the use of a thermostable polymerase such as Taq I polymerase and thermal cycling. In an alternative embodiment isothermal amplification is used.

Accordingly, the PCR reaction requires at least one PCR primer and a polymerase. Mesoscale PCR devices are described in U.S. Pat. Nos. 5,498,392 and 5,587,128, and WO 97/16561, incorporated by reference.

In a preferred embodiment the amplification is RT-PCR. Preferably the reaction includes either two-step RT-PCR or solid phase RT-PCR. In this embodiment RT-PCR can be performed using either solution phase primers or immobilized primers as described above. In this embodiment mRNA is reverse transcribed to cDNA and PCR is conducted by using DNA polymerase. Again PCR primers can be solution-phase or immobilized as described above.

In an additional preferred embodiment, re-amplification of cDNA (multiple-PCR system) is performed. cDNA synthesized from mRNA can be used more than once. Preferably, the cDNA is immobilized as this increases the stability of the cDNA. This allows reamplification of the same immobilized cDNA such that different or the same target sequences can be amplified multiple times. As noted above, amplification can use solution-phase primers or immobilized primers and detection of amplicons proceeds following hybridization of amplicons to the probe immobilized on the microsensor.

In a preferred embodiment the RT-PCR amplification is a high throughput RT-PCR system.

In a preferred embodiment, the amplification technique is LCR. The method can be run in two different ways; in a first embodiment, only one strand of a target sequence is used as a template for ligation; alternatively, both strands may be used. See generally U.S. Pat. Nos. 5,185,243 and 5,573,907; EP 0 320308B1; EP0336731 B1; EP0439 182B1; WO 90/01069; WO 89/12696; and WO 89/09835, and U.S. Ser. Nos. 60/078,102 and 60/073,011, all of which are incorporated by reference.

In a preferred amplification embodiment, the single-stranded target sequence comprises a first target domain and a second target domain. A first LCR primer and a second LCR primer nucleic acids are added, that are substantially complementary to its respective target domain and thus will hybridize to the target domains. These target domains may be directly adjacent, i.e. contiguous, or separated by a number of nucleotides. If they are non-contiguous, nucleotides are added along with means to join nucleotides, such as a polymerase, that will add the nucleotides to one of the primers. The two LCR primers are then covalently attached, for example using a ligase enzyme such as is known in the art. This forms a first hybridization complex comprising the ligated probe and the target sequence. This hybridization complex is then denatured (disassociated), and the process is repeated to generate a pool of ligated probes, i.e. amplicons. The ligated probes or amplicons are then detected with the probe immobilized on the microsensor.

In a preferred embodiment, LCR is done for two strands of a double-stranded target sequence. The target sequence is denatured, and two sets of primers are added: one set as outlined above for one strand of the target, and a separate set (i.e. third and fourth primer nucleic acids) for the other strand of the target. In a preferred embodiment, the first and third primers will hybridize, and the second and fourth primers will hybridize, such that amplification can occur. That is, when the first and second primers have been attached, the ligated product can now be used as a template, in addition to the second target sequence, for the attachment of the third and fourth primers. Similarly, the ligated third and fourth products will serve as a template for the attachment of the first and second primers, in addition to the first target strand. In this way, an exponential, rather than just a linear, amplification can occur.

Again, as outlined above, the detection of the LCR products can occur directly, in the case where one or both of the primers simply hybridize with a primer immobilized on the microsensor; hybridization is detected as described herein. Alternatively, detection of LCR products can occur indirectly using sandwich assays, through the use of additional probes; that is, the ligated products can serve as target sequences, and detection proceeds via hybridization to probes or primers immobilized on the surface of the microsensor.

In addition, the device may include other modules such as sample preparation chambers. In this embodiment, a crude sample is added to the sample treatment channel or chamber and is manipulated to prepare the sample for detection. The manipulated sample is removed from the sample treatment channel or chamber and added to the detection chamber. There may be additional functional elements into which the device fits; for example, a heating element may be placed in contact with the sample channel or chamber to effect reactions such as PCR. In some cases, a portion of the device may be removable; for example, the sample chamber may have a detachable detection chamber, such that the entire sample chamber is not contacted with the detection apparatus. See for example U.S. Pat. No. 5,603,351 and PCT US96/17116, hereby incorporated by reference.

In addition to different channels or chambers, the device may also include one or more flow cells or flow channels allowing sample movement between chambers. In addition to flow channels, there also may be inlet ports and outlet ports separating chambers. Such ports allow for samples to be contained in different chambers without cross-contamination.

In some embodiments the device also includes a pump mechanism that hydrodynamically pumps the samples through the device. Alternatively a vacuum device is used.

In a preferred embodiment, the microfluidic device can be made from a wide variety of materials, including, but not limited to, silicon such as silicon wafers, silcon dioxide, silicon nitride, glass and fused silica, gallium arsenide, indium phosphide, aluminum, ceramics, polyimide, quartz, plastics, resins and polymers including polymethylmethacrylate, acrylics, polyethylene, polyethylene terepthalate, polycarbonate, polystyrene and other styrene copolymers, polypropylene, polytetrafluoroethylene, superalloys, zircaloy, steel, gold, silver, copper, tungsten, molybdeumn, tantalum, KOVAR, KEVLAR, KAPTON, MYLAR, brass, sapphire, etc.

The microfluidic devices of the invention can be made in a variety of ways, as will be appreciated by those in the art. See for example WO96/39260, directed to the formation of fluid-tight electrical conduits; U.S. Pat. No. 5,747,169, directed to sealing; and EP 0637996 B1; EP 0637998 B1; WO96/39260; WO97/16835; WO98/13683; WO97/16561; WO97/43629; WO96/39252; WO96/15576; WO96/15450; WO97/37755; and WO97/27324; and U.S. Pat. Nos. 5,304,487; 5,071531; 5,061,336; 5,747,169; 5,296,375; 5,110,745; 5,587,128; 5,498,392; 5,643,738; 5,750,015; 5,726,026; 5,35,358; 5,126,022; 5,770,029; 5,631,337; 5,569,364; 5,135,627; 5,632,876; 5,593,838; 5,585,069; 5,637,469; 5,486,335; 5,755,942; 5,681,484; and 5,603,351, all of which are hereby incorporated by reference. Suitable fabrication techniques again will depend on the choice of substrate, but preferred methods include, but are not limited to, a variety of micromachining and microfabrication techniques, including film deposition processes such as spin coating, chemical vapor deposition, laser fabrication, photolithographic and other etching techniques using either wet chemical processes or plasma processes, embossing, injection molding, and bonding techniques (see U.S. Pat. No. 5,747,169, hereby incorporated by reference). In addition, there are printing techniques for the creation of desired fluid guiding pathways; that is, patterns of printed material can permit directional fluid transport.

In a preferred embodiment, the device is configured for handling a single sample that may contain a plurality of target analytes. That is, a single sample is added to the device and the sample may either be aliquoted for parallel processing for detection of the analytes or the sample may be processed serially, with individual targets being detected in a serial fashion.

In a preferred embodiment, the solid substrate is configured for handling multiple samples, each of which may contain one or more target analytes. In general, in this embodiment, each sample is handled individually; that is, the manipulations and analyses are done in parallel, with preferably no contact or contamination between them. Alternatively, there may be some steps in common; for example, it may be desirable to process different samples separately but detect all of the target analytes on a single detection array, as described below.

Thus, the multi-chamber devices of the invention include at least one microchannel or flow channel that allows the flow of sample from the sample inlet port to the other components or modules of the system. The collection of microchannels and wells is sometimes referred to in the art as a “mesoscale flow system”. As will be appreciated by those in the art, the flow channels may be configured in a wide variety of ways, depending on the use of the channel. For example, a single flow channel starting at the sample inlet port may be separated into a variety of different channels, such that the original sample is divided into discrete subsamples for parallel processing or analysis. Alternatively, several flow channels from different modules, for example the sample inlet port and a reagent storage module may feed together into a mixing chamber or a reaction chamber. As will be appreciated by those in the art, there are a large number of possible configurations; what is important is that the flow channels allow the movement of sample and reagents from one part of the device to another. For example, the path lengths of the flow channels may be altered as needed; for example, when mixing and timed reactions are required, longer and sometimes tortuous flow channels can be used; similarly, longer lengths for separation purposes may also be desirable.

In general, the microfluidic devices of the invention are generally referred to as “mesoscale” devices. The devices herein are typically designed on a scale suitable to analyze microvolumes, although in some embodiments large samples (e.g. cc's of sample) may be reduced in the device to a small volume for subsequent analysis. That is, “mesoscale” as used herein refers to chambers and microchannels that have cross-sectional dimensions on the order of 0.1 μm to 500 μm. The mesoscale flow channels and wells have preferred depths on the order of 0.1 μm to 100 μm, typically 2-50 μm. The channels have preferred widths on the order of 2.0 to 500 μm, more preferably 3-100 μm. For many applications, channels of 5-50 μm are useful. However, for many applications, larger dimensions on the scale of millimeters may be used. Similarly, chambers in the substrates often will have larger dimensions, on the scale of a few millimeters.

In addition to the flow channel system, the devices of the invention may be configured to include one or more of a variety of components, herein referred to as “modules”, that will be present on any given device depending on its use. These modules include, but are not limited to: sample inlet ports; sample introduction or collection modules; cell handling modules (for example, for cell lysis, cell removal, cell concentration, cell separation or capture, cell fusion, cell growth, etc.); separation modules, for example, for electrophoresis, gel filtration, sedimentation, etc.); reaction modules for chemical or biological alteration of the sample, including amplification of the target analyte (for example, when the target analyte is nucleic acid, amplification techniques are useful, including, but not limited to polymerase chain reaction (PCR), ligase chain reaction (LCR), strand displacement amplification (SDA), chemical, physical or enzymatic cleavage or alteration of the target analyte, or chemical modification of the target; fluid pumps; fluid valves; heating modules; storage modules for assay reagents; mixing chambers; and detection modules.

In a preferred embodiment, the devices of the invention include at least one sample inlet port for the introduction of the sample to the device. This may be part of or separate from a sample introduction or collection module; that is, the sample may be directly fed in from the sample inlet port to a separation chamber, or it may be pretreated in a sample collection well or chamber. Alternatively, for example, when there is a single chamber, the sample inlet port may be configured such that samples are introduced into the single chamber for amplification and/or detection.

In a preferred embodiment, the devices of the invention include a sample collection module, which can be used to concentrate or enrich the sample if required; for example, see U.S. Pat. No. 5,770,029, including the discussion of enrichment channels and enrichment means.

In a preferred embodiment, the devices of the invention include a cell handling module. This is of particular use when the sample comprises cells that either contain the target analyte or that are removed in order to detect the target analyte. Thus, for example, the detection of particular antibodies in blood can require the removal of the blood cells for efficient analysis, or the cells must be lysed prior to detection. In this context, “cells” include viral particles that may require treatment prior to analysis, such as the release of nucleic acid from a viral particle prior to detection of target sequences. In addition, cell handling modules may also utilize a downstream means for determining the presence or absence of cells. Suitable cell handling modules include, but are not limited to, cell lysis modules, cell removal modules, cell concentration modules, and cell separation or capture modules. In addition, as for all the modules of the invention, the cell handling module is in fluid communication via a flow channel with at least one other module of the invention.

In a preferred embodiment, the cell handling module includes a cell lysis module. As is known in the art, cells may be lysed in a variety of ways, depending on the cell type. In one embodiment, as described in EP 0 637 998 B1 and U.S. Pat. No. 5,635,358, hereby incorporated by reference, the cell lysis module may comprise cell membrane piercing protrusions that extend from a surface of the cell handling module. As fluid is forced through the device, the cells are ruptured. Similarly, this may be accomplished using sharp edged particles trapped within the cell handling region. Alternatively, the cell lysis module can comprise a region of restricted cross-sectional dimension, which results in cell lysis upon pressure.

In a preferred embodiment, the cell lysis module comprises a cell lysing agent, such as detergents, NaOH, enzymes, proteinase K, guanidinium HCL, etc. In some embodiments, for example for blood cells, a simple dilution with water or buffer can result in hypotonic lysis. The lysis agent may be solution form, stored within the cell lysis module or in a storage module and pumped into the lysis module. Alternatively, the lysis agent may be in solid form, that is taken up in solution upon introduction of the sample. Temperature or mixing may also be applied.

The cell lysis module may also include, either internally or externally, a filtering module for the removal of cellular debris as needed. This filter may be microfabricated between the cell lysis module and the subsequent module to enable the removal of the lysed cell membrane and other cellular debris components; examples of suitable filters are shown in EP 0 637 998 B 1, incorporated by reference.

In one embodiment of sample preparation, cells are placed or distributed on a filter membrane evenly and a lysis buffer is passed through the cell layer on the filter membrane without mechanical homogenization of the cells. This can be performed in a sample preparation chamber as described above. Alternatively, it may be performed prior to addition of the sample to the chamber.

In the above, the cell lysate can be passed through the membrane of the filter plate with the aid of force generated by means of centrifugation, vacuum, or positive pressure. The filter or membrane of the filter plate includes, but is not limited to, glass fiber, polypropylene or polyolefine mesh, wool, and other membranes which have a pore size such that target cells can be trapped without any leakage of cells from the membrane, but cytosolic mRNA can pass through. For example, using glass fiber (Grade 934AH, Cambridge Technology, Inc. Watertown, Mass.) or Whatman GFIF grade glass fiber membrane, most of cultured cells and blood leukocyte can be trapped. In the above, glass fiber plates are preferable.

The lysis buffer may include a detergent for dissolving cell membranes, RNase inhibitor for inhibiting RNase activity or deactivating or destroying RNase, and pH control agent and salt for hybridization. The isolated target sample can then be analyzed as described herein

Accordingly, a rapid, inexpensive, high throughput, and easily automated system can be realized.

In a preferred embodiment, the cell handling module includes a cell separation or capture module. This embodiment utilizes a cell capture region comprising binding sites capable of reversibly binding a cell surface molecule to enable the selective isolation (or removal) of a particular type of cell from the sample population. These binding moieties may be immobilized either on the surface of the module or on a particle trapped within the module by physical absorption or by covalent attachment. Suitable binding moieties will depend on the cell type to be isolated or removed, and generally includes antibodies and other binding ligands, such as ligands for cell surface receptors, etc. Thus, a particular cell type may be removed from a sample prior to further handling, or the assay is designed to specifically bind the desired cell type, wash away the non-desirable cell types, followed by either release of the bound cells by the addition of reagents or solvents, physical removal (i.e. higher flow rates or pressures), or even in situ lysis.

Alternatively, a cellular “sieve” can be used to separate cells on the basis of size or shape. This can be done in a variety of ways, including protrusions from the surface that allow size exclusion, a series of narrowing channels, or a diafiltration type setup.

In a preferred embodiment, the cell handling module includes a cell removal module. This may be used when the sample contains cells that are not required in the assay. Generally, cell removal will be done on the basis of size exclusion as for “sieving”, above, with channels exiting the cell handling module that are too small for the cells; filtration and centrifugation may also be done.

In a preferred embodiment, the cell handling module includes a cell concentration module. As will be appreciated by those in the art, this is done using “sieving” methods, for example to concentrate the cells from a large volume of sample fluid prior to lysis, or centrifugation.

In a preferred embodiment, the devices of the invention include a separation module. Separation in this context means that at least one component of the sample is separated from other components of the sample. This can comprise the separation or isolation of the target analyte, or the removal of contaminants that interfere with the analysis of the target analyte, depending on the assay.

In a preferred embodiment, the separation module includes chromatographic-type separation media such as absorptive phase materials, including, but not limited to reverse phase materials (C₈ or C₁₈ coated particles, etc.), ion-exchange materials, affinity chromatography materials such as binding ligands, etc. See U.S. Pat. No. 5,770,029.

In a preferred embodiment, the separation module utilizes binding ligands, as is generally outlined herein for cell separation or analyte detection.

When the sample component bound by the binding ligand is the target analyte, it may be released for detection purposes if necessary, using any number of known techniques, depending on the strength of the binding interaction, including changes in pH, salt concentration, temperature, etc. or the addition of competing ligands, etc.

In a preferred embodiment, the separation module includes an electrophoresis module, as is generally described in U.S. Pat. Nos. 5,770,029; 5,126,022; 5,631,337; 5,569,364; 5,750,015, and 5,135,627, all of which are hereby incorporated by reference. In electrophoresis, molecules are primarily separated by different electrophoretic mobilities caused by their different molecular size, shape and/or charge. Microcapillary tubes have recently been used for use in microcapillary gel electrophoresis (high performance capillary electrophoresis (HPCE)). One advantage of HPCE is that the heat resulting from the applied electric field is efficiently disippated due to the high surface area, thus allowing fast separation. The electrophoresis module serves to separate sample components by the application of an electric field, with the movement of the sample components being due either to their charge or, depending on the surface chemistry of the microchannel, bulk fluid flow as a result of electroosmotic flow (EOF).

As will be appreciated by those in the art, the electrophoresis module can take on a variety of forms, and generally comprises an electrophoretic microchannel and associated electrodes to apply an electric field to the electrophoretic microchannel. Waste fluid outlets and fluid reservoirs are present as required.

The electrodes comprise pairs of electrodes, either a single pair, or, as described in U.S. Pat. Nos. 5,126,022 and 5,750,015, a plurality of pairs. Single pairs generally have one electrode at each end of the electrophoretic pathway. Multiple electrode pairs may be used to precisely control the movement of sample components, such that the sample components may be continuously subjected to a plurality of electric fields either simultaneously or sequentially. Such a system is outlined in 5,858,195, incorporated herein by reference

In a preferred embodiment, electrophoretic gel media may also be used. By varying the pore size of the media, employing two or more gel media of different porosity, and/or providing a pore size gradient, separation of sample components can be maximized. Gel media for separation based on size are known, and include, but are not limited to, polyacrylamide and agarose. One preferred electrophoretic separation matrix is described in U.S. Pat. No. 5,135,627, hereby incorporated by reference, that describes the use of “mosaic matrix”, formed by polymerizing a dispersion of microdomains (“dispersoids”) and a polymeric matrix. This allows enhanced separation of target analytes, particularly nucleic acids. Similarly, U.S. Pat. No. 5,569,364, hereby incorporated by reference, describes separation media for electrophoresis comprising submicron to above-micron sized cross-linked gel particles that find use in microfluidic systems. U.S. Pat. No. 5,631,337, hereby incorporated by reference, describes the use of thermoreversible hydrogels comprising polyacrylamide backbones with N-substituents that serve to provide hydrogen bonding groups for improved electrophoretic separation. See also U.S. Pat. Nos. 5,061,336 and 5,071,531, directed to methods of casting gels in capillary tubes.

In a preferred embodiment, the devices of the invention include at least one fluid pump. Pumps generally fall into two categories: “on chip” and “off chip”; that is, the pumps (generally syringe pumps or electrode based pumps) can be contained within the device itself, or they can be contained on an apparatus into which the device fits, such that alignment occurs of the required flow channels to allow pumping of fluids.

In a preferred embodiment, the devices of the invention include at least one fluid valve that can control the flow of fluid into or out of a module of the device. A variety of valves are known in the art. For example, in one embodiment, the valve may comprise a capillary barrier, as generally described in PCT US97/07880, incorporated by reference. In this embodiment, the channel opens into a larger space designed to favor the formation of an energy minimizing liquid surface such as a meniscus at the opening. Preferably, capillary barriers include a dam that raises the vertical height of the channel immediated before the opening into a larger space such a chamber. In addition, as described in U.S. Pat. No. 5,858,195, incorporated herein by reference, a type of “virtual valve” can be used.

In a preferred embodiment, the devices of the invention include sealing ports, to allow the introduction of fluids, including samples, into any of the modules of the invention, with subsequent closure of the port to avoid the loss of the sample.

Once made, the device of the invention finds use in a variety of applications. Preferred applications include forensics, mutation detection, microorganism or pathogen detection and the like.

As to forensics, the identification of individuals at the level of DNA sequence variation offers a number of practical advantages over such conventional criteria as fingerprints, blood type, or physical characteristics. In contrast to most phenotypic markers, DNA analysis readily permits the deduction of relatedness between individuals such as is required in paternity testing. Genetic analysis has proven highly useful in bone marrow transplantation, where it is necessary to distinguish between closely related donor and recipient cells. Two types of probes are now in use for DNA fingerprinting by DNA blots. Polymorphic minisatellite DNA probes identify multiple DNA sequences, each present in variable forms in different individuals, thus generating patterns that are complex and highly variable between individuals. VNTR probes identify single sequences in the genome, but these sequences may be present in up to 30 different forms in the human population as distinguished by the size of the identified fragments. The probability that unrelated individuals will have identical hybridization patterns for multiple VNTR or minisatellite probes is very low. Much less tissue than that required for DNA blots, even single hairs, provides sufficient DNA for a PCR-based analysis of genetic markers. Also, partially degraded tissue may be used for analysis since only small DNA fragments are needed. Forensic DNA analyses will eventually be carried out with polymorphic DNA sequences that can be studied by simple automatable assays such as OLA. For example, the analysis of 22 separate gene sequences, each one present in two different forms in the population, could generate 1010 different outcomes, permitting the unique identification of human individuals. That is, the unique pattern of mass increases as a result of detecting unique genes, exon/intron boundaries, SNPs, mRNA and the like results in the unique identification of an individual.

In another preferred embodiment the device finds use in tumor diagnostics. The detection of viral or cellular oncogenes is another important field of application of nucleic acid diagnostics. Viral oncogenes (v-oncogenes) are transmitted by retroviruses while their cellular counterparts (c-oncogenes) are already present in normal cells. The cellular oncogenes can, however, be activated by specific modifications such s point mutations (as in the c-K-ras oncogene in bladder carcinoma and in colorectal tumors), promoter induction, gene amplification (as in the N-myc oncogene in the case of neuroblastoma) or the rearrangement of chromosomes (as in the translocation of the c-abl oncogene from chromosome 9 to chromosome 22 in the case of chronic myeloid leukemia). Each of the activation processes leads, in conjunction with additional degenerative processes, to an increased and uncontrolled cell growth. The so-called “recessive oncogenes” which must be inactivated for the formation of a tumor (as in the retinobiastoma (Rb gene and the osteosarcoma can also be detected with the help of DNA probes. Using probes against immunoglobulin genes and against T-cell receptor genes, the detection of B-cell lymphomas and lymphoblastic leukemia is possible. As such, the invention provides a method and device for diagnosing tumor types. Nucleic acid probes or antibodies directed to various tumor markers are used as bioactive agents for the detection of tumor markers.

In an additional preferred embodiment the device finds use in transplantation analyses. The rejection reaction of transplanted tissue is decisively controlled by a specific class of histocompatibility antigens (HLA). They are expressed on the surface of antigen-presenting blood cells, e.g., macrophages. The complex between the HLA and the foreign antigen is recognized by T-helper cells through corresponding T-cell receptors on the cell surface. The interaction between HLA, antigen and T-cell receptor triggers a complex defense reaction which leads to a cascade-like immune response on the body. The recognition of different foreign antigens is mediated by variable, antigen-specific regions of the T-cell receptor-analogous to the antibody reaction. In a graft rejection, the T-cells expressing a specific T-cell receptor which fits to the foreign antigen, could therefore be eliminated from the T-cell pool. Such analyses are possible by the identification of antigen-specific variable DNA sequences which are amplified by PCR and hence selectively increased. The specific amplification reaction permits the single cell-specific identification of a specific T-cell receptor. Similar analyses are presently performed for the identification of auto-immune disease like juvenile diabetes, arteriosclerosis, multiple sclerosis, rheumatoid arthritis, or encephalomyelitis.

In an additional preferred embodiment the device finds use in genome diagnostics. Four percent of all newborns are born with genetic defects; of the 3,500 hereditary diseases described which are caused by the modification of only a single gene, the primary molecular defects are only known for about 400 of them. Hereditary diseases have long since been diagnosed by phenotypic analyses (anamneses, e.g., deficiency of blood: thalassemias), chromosome analyses (karyotype, e.g., mongolism: trisomy 21) or gene product analyses (modified proteins, e.g., phenylketonuria: deficiency of the phenylalanine hydroxylase enzyme resulting in enhanced levels of phenylpyruvic acid). The additional use of nucleic acid detection methods considerably increases the range of genome diagnostics.

In the case of certain genetic diseases, the modification of just one of the two alleles is sufficient for disease (dominantly transmitted monogenic defects); in many cases, both alleles must be modified (recessively transmitted monogenic defects). In a third type of genetic defect, the outbreak of the disease is not only determined by the gene modification but also by factors such as eating habits (in the case of diabetes or arteriosclerosis) or the lifestyle (in the case of cancer). Very frequently, these diseases occur in advanced age. Diseases such as schizophrenia, manic depression or epilepsy should also be mentioned in this context; it is under investigation if the outbreak of the disease in these cases is dependent upon environmental factors as well as on the modification of several genes in different chromosome locations. Using direct and indirect DNA analysis, the diagnosis of a series of genetic diseases has become possible: sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, Huntington's chorea.

In an additional preferred embodiment the device finds use in pharmacogenomics. Pharmacogenomics has evolved from the academic science into an important tool for drug research and development. Accordingly, a new paradigm has evolved to target drug to patients with a specific genetic profile that predicts a favorable response to therapy. Different genes expression level of specific SNP's into certain genes can be useful for the treatment of cancer, diabetes and cardiovascular disease. Those candidate genes can be used to profile patients and their disease to allow for optimal treatment based on the presence or absence of specific genetic polymorphisms. By focusing on loci that appear to predict the onset of disease, it is the hope that pharmaceutical companies will intervene with new compounds designed to halt the progression of disease. When pharmacogenomics is integrated into drug research it allows pharmaceutical companies to stratify patient populations based on genetic background. During drug development, these same markers can be used to link efficacy or disease susceptibility to new pharmaceutical compounds. To be able to measure such changes in either single gene, many genes either as SNP or simple changes in expression level it requires a method as described to which may be utilized to overcome the challenges of modifying biological material such as DNA before measurement, enhance sample number throughput in a wide variety of based assays and overcome the used of highly specialized and expensive equipment.

In an additional preferred embodiment the device finds use in infectious disease. The application of recombinant DNA methods for diagnosis of infectious diseases has been most extensively explored for viral infections where current methods are cumbersome and results are delayed. In situ hybridization of tissues or cultured cells has made diagnosis of acute and chronic herpes infection possible. Fresh and fomalin-fixed tissues have been reported to be suitable for detection of papillomavirus in invasive cervical carcinoma and in the detection of HIV, while cultured cells have been used for the detection of cytomegalovirus and Epstein-Barr virus. The application of recombinant DNA methods to the diagnosis of microbial diseases has the potential to replace current microbial growth methods if cost-effectiveness, speed, and precision requirements can be met. Clinical situations where recombinant DNA procedures have begun to be applied include the identification of penicillin-resistant Neisseria gonorrhea by the presence of a transposon, the fastidiously growing chlamydia, microbes in foods; and simple means of following the spread of an infection through a population. The worldwide epidemiological challenge of diseases involving such parasites as leishmania and plasmodia is already being met by recombinant methods.

In an additional preferred embodiment the device finds use in gene expression analysis. One of the inventions disclosed herein is a high throughput method for measuring the expression of numerous genes (1-100) in a single measurement. The method also has the ability to be done in parallel with greater than one hundred samples per process. The method is applicable to drug screening, developmental biology, molecular medicine studies and the like. Thus, within one aspect of the invention methods are provided for analyzing the pattern of gene expression from a selected biological sample, comprising the steps of (a) exposing nucleic acids from a biological sample, (b) combining the exposed nucleic acids with one or more selected nucleic acid probes each located on a particular microsensor, under conditions and for a time sufficient for said probes to hybridize to said nucleic acids, wherein the hybridization correlative with a particular nucleic acid probe and detectable by the DNA-amplification-microsensor technology.

In additional preferred embodiments the device finds use in detection of micro-organisms, specific gene expression or specific sequences in nucleic acid. The use of DNA probes in combination with the DNA-amplification-microsensor technology can be used to detect the presence or absence of micro-organisms in any type of sample or specimen. Detectable nucleic acid can include mRNA, genomic DNA, plasmid DNA or RNA, rRNA viral DNA or RNA.

In an additional preferred embodiment the device finds use in mutation detection techniques. The detection of diseases is increasingly important in prevention and treatments. While multi factorial diseases are difficult to devise genetic tests for, more than 200 known human disorders are caused by a defect in a single gene, often a change of a single amino acid residue (Olsen, Biotechnology: An industry comes of age, National Academic Press, 1986). Many of these mutations result in an altered amino acid that causes a disease state.

Those point mutations are often called single-nucleotide polymorphisms (SNP) or cSNP when the point mutation are located in the coding region of a gene.

Sensitive mutation detection techniques offer extraordinary possibilities for mutation screening. For example, analyses may be performed even before the implantation of a fertilized egg (Holding and Monk, Lancet 3:532, 1989). Increasingly efficient genetic tests may also enable screening for oncogenic mutations in cells exfoliated from the respiratory tract or the bladder in connection with health checkups (Sidransky et al., Science 252:706, 1991). Also, when an unknown gene causes a genetic disease, methods to monitor DNA sequence variants are useful to study the inheritance of disease through genetic linkage analysis. However, detecting and diagnosing mutations in individual genes poses technological and economic challenges. Several different approaches have been pursued, but none are both efficient and inexpensive enough for truly widescale application.

Mutations involving a single nucleotide can be identified in a sample by physical, chemical, or enzymatic means. Generally, methods for mutation detection may be divided into scanning techniques, which are suitable to identify previously unknown mutations, and techniques designed to detect, distinguish, or quantitate known sequence variants, it is within that last described this invention has its strong advances compared to known status of the art technology.

Mutations are a single-base pair change in genomic DNA. Within the context of this invention, most such changes are readily detected by hybridization with oligonucleotides that are complementary to the sequence in question. In the system described here, two oligonucleotides are employed to detect a mutation. One oligonucleotide possesses the wild-type sequence and the other oligonucleotide possesses the mutant sequence. When the two oligonucleotides are used as probes on a wild-type target genomic sequence, the wild-type oligonucleotide will form a perfectly based paired structure and the mutant oligonucleotide sequence will form a duplex with a single base pair mismatch.

As discussed above, a 6 to 7° C. difference in the Tm of a wild type versus mismatched duplex permits the ready identification or discrimination of the two types of duplexes. To effect this discrimination, hybridization is performed at the Tm of the mismatched duplex in the respective hybotropic solution. The extent of hybridization is then measured for the set of oligonucleotide probes. When the ratio of the extent of hybridization of the wild-type probe to the mismatched probe is measured, a value to 10/1 to greater than 20/1 is obtained. These types of results permit the development of robust assays for mutation detection.

Other highly sensitive hybridization protocols may be used. The methods of the present invention enable one to readily assay for a nucleic acid containing a mutation suspected of being present in cells, samples, etc., i.e., a target nucleic acid. The “target nucleic acid” contains the nucleotide sequence of deoxyribonucleic acid (DNA) or ribonucleic acid (RNA) whose presence is of interest, and whose presence or absence is to be detected for in the hybridization assay. The hybridization methods of the present invention may also be applied to a complex biological mixture of nucleic acid (RNA and/or DNA). Such a complex biological mixture includes a wide range of eucaryotic and procaryotic cells, including protoplasts; and/or other biological materials which harbor polynucleotide nucleic acid. The method is thus applicable to tissue culture cells, animal cells, animal tissue, blood cells (e.g., reticulocytes, lymphocytes), plant cells, bacteria, yeasts, viruses, mycoplasmas, protozoa, fungi and the like. By detecting a specific hybridization between nucleic acid probes of a known source the specific presence of a target nucleic acid can be established.

An exemplary hybridization assay protocol for detecting a target nucleic acid in a complex population of nucleic acids is described as follows: A probe containing the SNP at the 3′ end is immobilized on one micro-cantilever at it's 5′ end (probe 1). Within the surroundings of the first micro-cantilever a second micro-cantilever is immobilized with a probe having the wild type sequence (probe 2). Two primer are designed for PCR amplification of a PCR product containing the potential SNP site. Normally the probe sites are located close to one of the primer sites. The following events may occur simultaneously in the chamber: 1) DNA amplification of target nucleic acid molecule in solution using the two above primers 2) hybridization of amplified target nucleic acid molecule to the probe 1 and probe 2 immobilized on two different cantilevers. The target nucleic acid molecules are capable of hybridizing to the 3′ region of the immobilized probe sequence, to thereby form a hybridization complex that has a 3′ terminus; 3) 3′ extension of the DNA strand hybridized to the immobilized probe on the surface of the cantilever to form a modified primer. If the DNA tested has the SNP site, probe 1 will hybridize more efficiently to the DNA compared to probe 2 where a 3′ mismatch will inhibit the 3′ extension reaction of the DNA strand hybridized to the immobilized probe on the surface of the cantilever. If the DNA tested does not contain SNP site (wild type), probe 2 will hybridize more efficiently to the DNA compared to probe 1 where a 3′ mismatch will inhibit the 3′ extension reaction of the DNA strand hybridized to the immobilized probe on the surface of the cantilever. Those observations can be directly observed due to different mechanical stress detection levels.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference.

### EXAMPLES

### Example 1

Detection of probes being immobilized to a gold-coated micro-cantilever surface (programming the cantilever chip).

The measuring and reference micro-cantilevers are multilayer structures with a top silicon nitride layer and a bottom silicon layer. The measuring cantilever is coated on one side with 60 nm of gold for thiol-modified DNA-probe immobilization, whereas the reference cantilever is left uncoated. Initially, all immobilization and hybridization steps were tested on pieces of silicon, silicon nitride and gold-coated silicon (data not shown).

Alternatively each of the cantilever based sensors is coated with a thio-modified oligonucleotide and then inserted into the chamber. Using capillary micro tubes it is possible to coat an array (one that one cantilever) of cantilevers with one than one thio-modified DNA oligo.

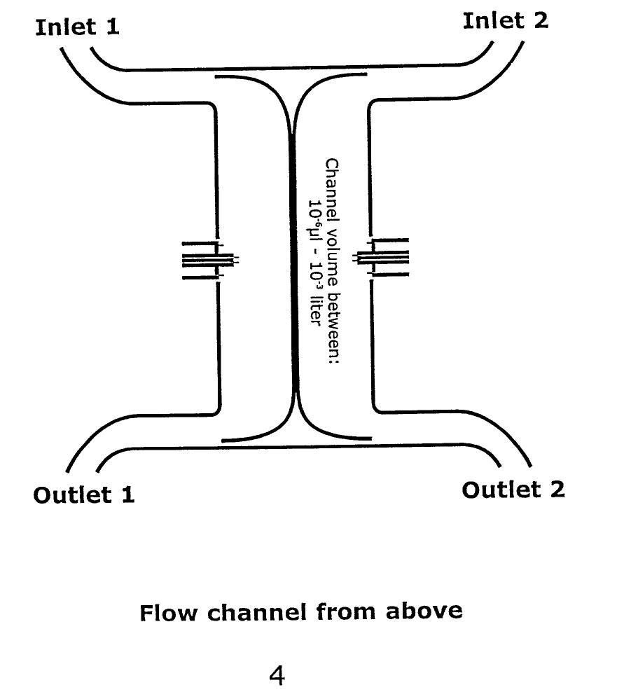

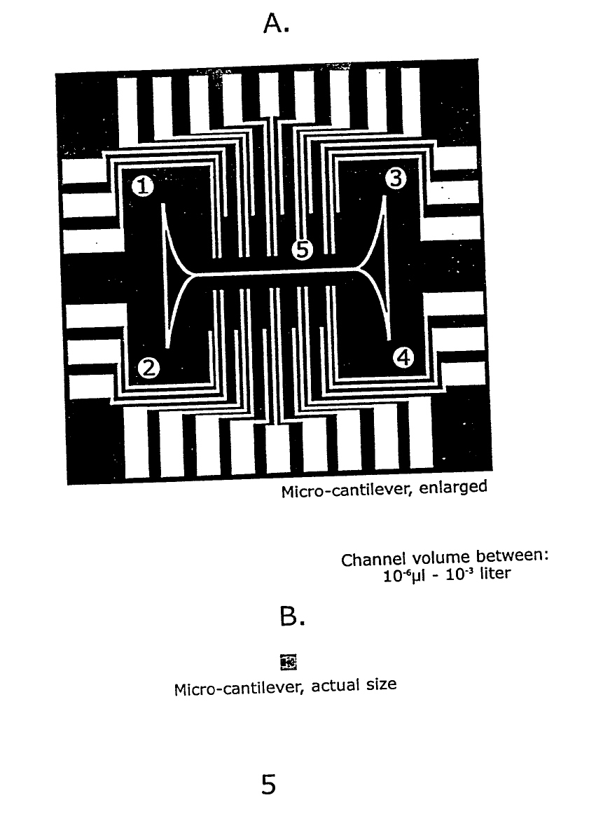

For in situ studies of immobilization and hybridization the cantilever-based sensor is placed in a x μl flow cell having integrated 2 micro-cantilevers as illustrated at FIG. 4, where the sensor signal for the micro-cantilever (1 signal and 1 reference) can be separate and continuously recorded. Syringe pumps and an automated valve control the liquid flow through the cell. The experiments are performed at room temperature and the flow rate is in all experiments x μl/min Prior to immobilization of the thiol-modified DNA-probe, the gold surface is cleaned by pumping a diluted aqua regia, AR, solution through the cell, followed by a rinse in D1 water. After preparation of the sensor's gold receptor surface, two different probes solution of thiol-modified DNA probes is pumped through the system while the time dependent cantilever response is being monitored.

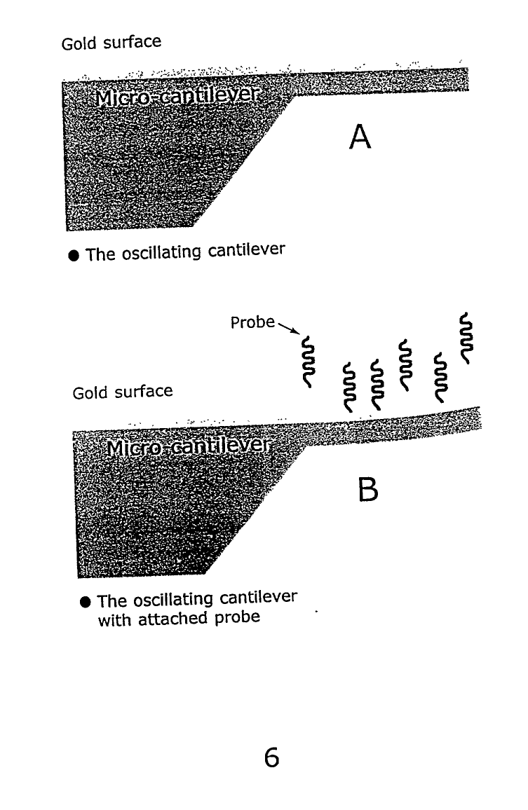

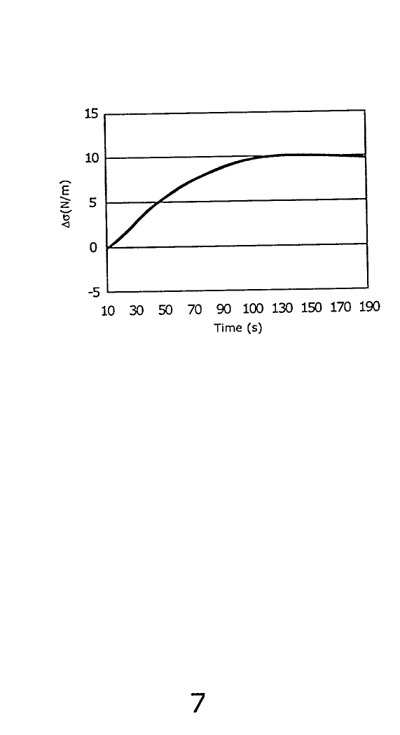

Due to the small channel dimensions, the flow is laminar with negligible influence of inertial forces. This makes it possible to inject to different DNA probes without mixing of the two probes. The thickness of the sample flow can be precisely adjusted by variation of the flow rate of the two probes solutions (FIG. 4). Thiols are known to form self-assembled monolayers on gold, and the thiol-modified DNA-oligos are therefore expected to immobilise on the gold-coated cantilever as illustrated at FIG. 6. It is observed that the cantilever reacts strongly when exposed to the thiol-modified DNA probes, and after approximately 100 s the cantilever signal stabilizes, as illustrated at FIG. 7. The time dependence of the layer formation is modeled by two diffusion-limited Langmuir isotherms, in which the number of adsorption sites is fixed and the concentration of thiol-probes is assumed constant. The two isotherms are believed to reflect the desorption of impurities and the successive adsorption of thiol-probe. Since the stress curves follow Langmuir model characteristics we conclude that the surface stress is proportional to the number of adsorbed molecules.

Cantilever Sensor

The cantilever-based sensor is operated at a supply voltage of 2 V in all experiments. The cantilevers are 150 μm long, 40 μm wide and 1.3 μm thick and have a surface stress sensitivity of ΔR/Rσ⁻¹=4.4×10⁻⁴ N⁻¹m, where ΔR/R is the relative change in the resistance of the integrated piezoresistor. The sensor has a minimum detectable surface stress change of approximately 5×10⁻³ Nm⁻¹. The gauge factor K of the piezoresisitve material is defined as ΔR/R=Kε, where ε is the induced strain in the piezoresistor. Our piezoresistors are defined in poly-silicon and have a gauge factor of approximately 30. For measurements in liquid the electrical wires on the cantilever-based sensor as well as bonding wires to a ceramic substrate on which the probe is mounted need to be protected by an insulating material. For this purpose we have used a vacuum sealing wax. The wax is melted and the chip and ceramic substrate are immersed into the liquid wax using a micromanipulator. By this method one can coat the body of the sensor chip without coating the cantilevers.

Alternatively, the cantilever is electrically isolated from the substrate by a thin layer of silicon dioxide.

Before immobilization, the gold receptor surface of the sensor is etched in a mixture of HCL and HNO₃ (3:1, 25%), also called aqua regia (AR). This solution etches gold at a rate of 20 Å/min at room temperature. A cleaning procedure consisting of a 30 s gold etch followed by a thorough rinse in D1 water has proven to yield surfaces with good immobilization quality. Untreated gold surfaces as well as AR cleaned surfaces have been tested using fluorescent marked DNA probes, and the number of immobilised probes increases significantly when the AR clean is applied (data not shown).

DNA-probe preparation: Two DNA probes for measure the wild type Cystic Fibrosis gene and the AF508 mutation of the Cystic Fibrosis were synthesised (DNA Technology, Aarhus, Denmark), both capture DNA-probe being 5 thiol modified.

TABLE I
Capture probe wild type = PROBE_{wCF} and Capture
probe ΔF508 mutation = PROBE_{ΔCF}

| | |
|---|---|
| PROBE_{wC} | 5′DMT-S-(CH2)₁₂CCATTAAAGAAAATATCATCTT-3′ |
| PROBE_{ΔCF} | 5′DMT-S- |
| | (CH1)₁₂GCACCATTAAAGAAAATATCATCGG-3′ |

The two probes (wCF and ACF) are injected into the reaction chamber (FIG. 4, 5), at a concentration of 5 mM of each DNA probe and with the same flow rate (25 μl/min). The observed rate constant of the Langmuir function associated with the adsorption reaction is 3.7×10³ Mol⁻¹s⁻¹ and is comparable to a previously reported rate constant of 2.1×10³ Mol⁻¹s⁻¹ for double stranded thiol-modified DNA investigated by a quartz crystal resonator (Yang, M et al Langmuir, 14, 6121-6129,1998).

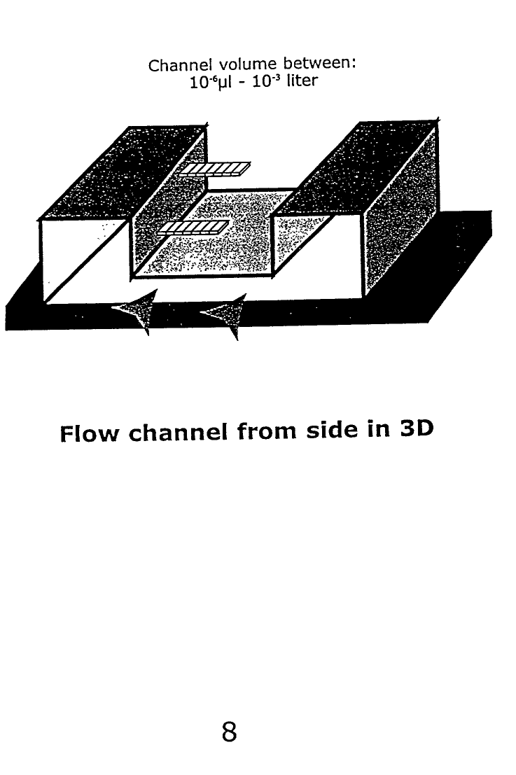

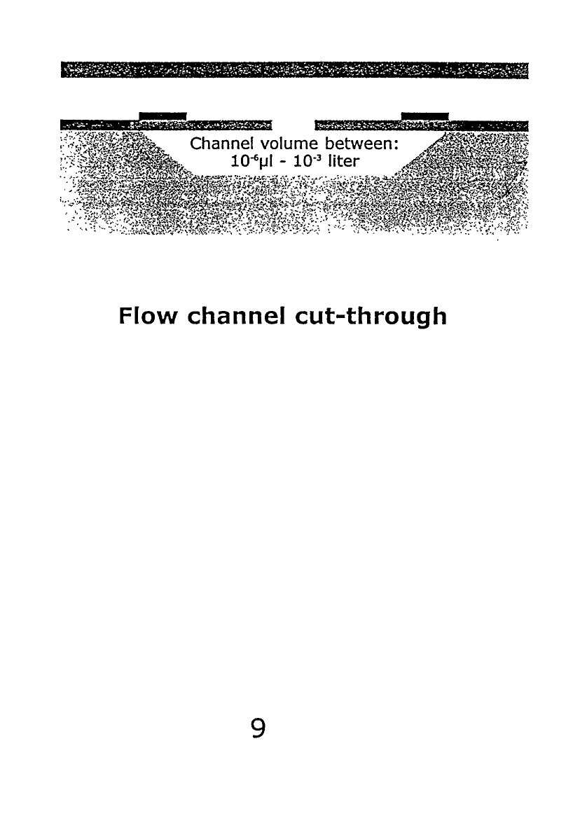

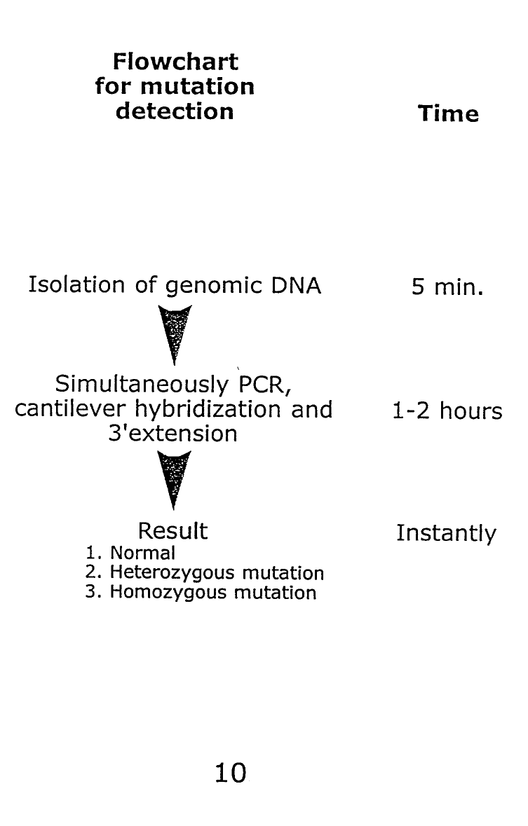

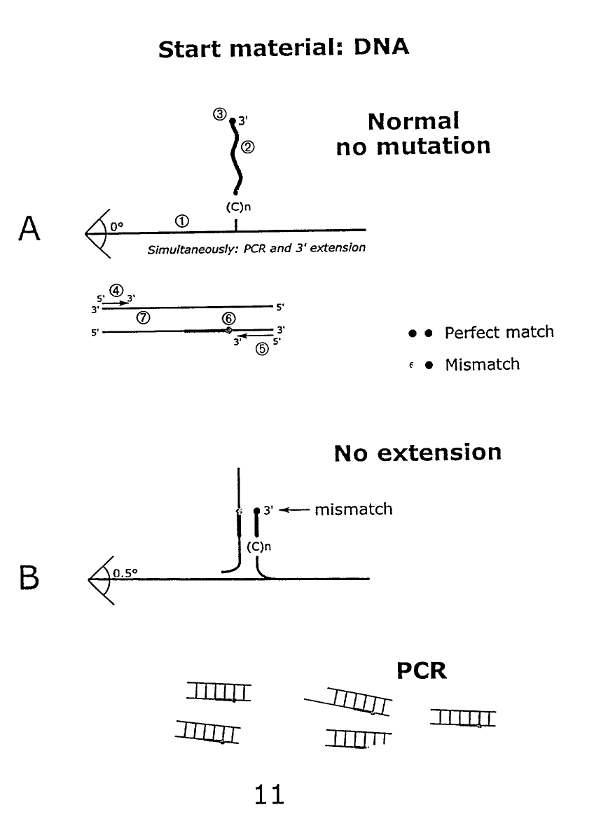

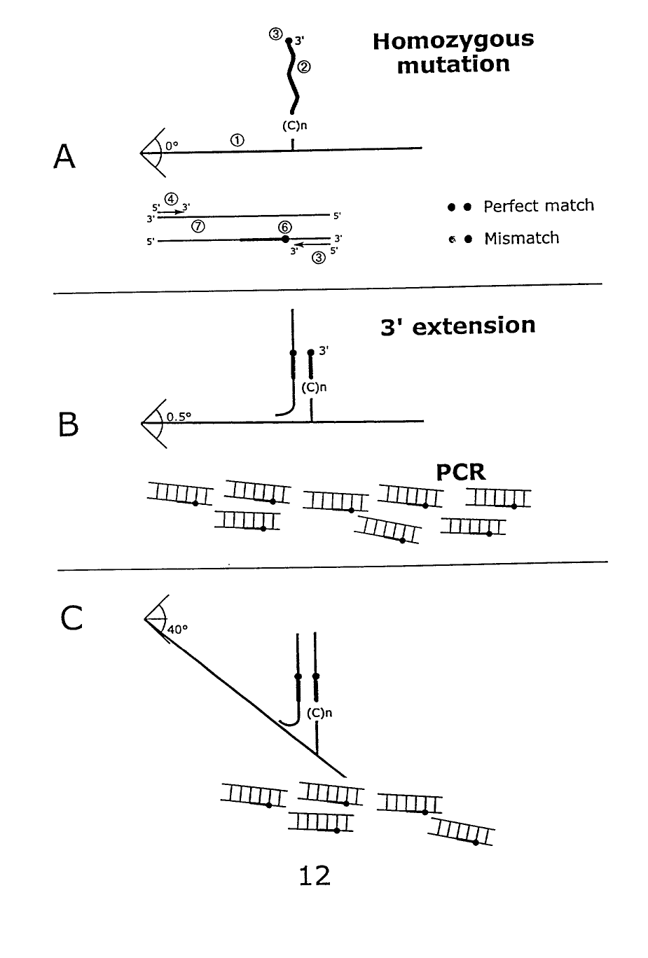

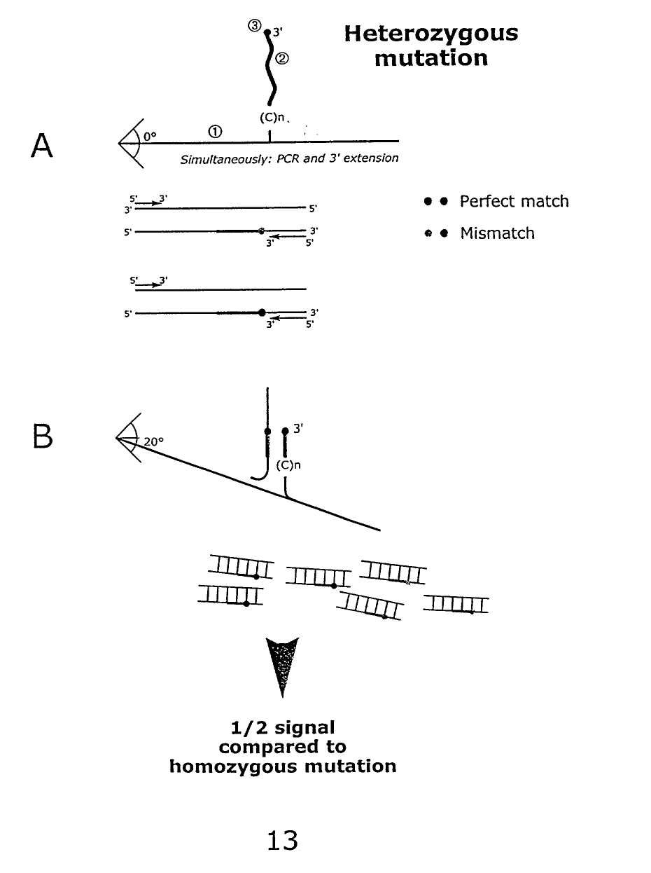

Each of the two gold coated micro-cantilever having either the wCF or the ACF immobilized DNA probes on the gold surface on the micro-cantilever gave surface stress changes of approximately 10 N/m for the wCF DNA probe illustrated in FIG. 7 and 8 N/m for the ΔCF DNA probe (data not shown).

We interpret the micro-cantilever response as being due to the formation of a completed layer of thiol-modified DNA-probes on the gold-coated cantilever. The surface stress change associated with the layer formation is approximately 8-10 N/m and the stress is tensile. That is, the formed DNA-probe layer contracts with respect to the cantilever substrate, causing the cantilever to bend towards the gold coated surface. The nature of the stress formation may be linked to intermolecular attractive forces, caused by base pairing between neighboring DNA-probes or caused by hydrophobic interactions.

### Example 2

Detection of the AF508 mutation of the Cystic Fibrosis gene (CFTR) using the PCR based micro-cantilevers as a sensor.

In USA the Cystic Fibrosis (CF) affects approximately 30,000 children and young adults. It occurs in approximately one of every 3,200 live Caucasian births (in one of every 3,900 live births of all Americans). There are about 1,000 new cases of CF diagnosed each year. Most individuals are diagnosed by the age of three; however, nearly 8 percent of all newly diagnosed cases are 18 or older. According to the CF Foundation s National Patient Registry, one half of all individuals with CF live to the age of 31; however, one half do not. One in 31 Americans (one in 28 Caucasians)-more than 10 million people is an unknowing, symptom less carrier of the defective gene. An individual must inherit a defective copy of the CF gene-one from each parent to have cystic fibrosis. Each time two carriers conceive a child, there is a 25 percent chance that the child will have CF; a 50 percent chance that the child will be a carrier; and a 25 percent chance that the child will be a non-carrier. CF has a variety of symptoms. The most common are: very salty-tasting skin; persistent coughing, wheezing or pneumonia; excessive appetite but poor weight gain; and bulky stools. The basic defect in CF cells is the faulty transport of sodium and chloride (salt) within epithelial cells-which line organs such as the lungs and pancreas-to their outer surfaces. CF causes the body to produce abnormally thick, sticky mucus. This abnormal mucus clogs the lungs and leads to fatal infections. The thick CF mucus also obstructs the pancreas, preventing enzymes from reaching the intestines to digest food. The treatment of CF depends upon the stage of the disease and which organs are involved. One means of treatment, postural drainage (also called chest physical therapy [CPT]), requires vigorous percussion (by using cupped hands) on the back and chest to dislodge the thick mucus from the lungs. Antibiotics are also used to treat lung infections and are administered intravenously, via pills, and/or medicated vapors which are inhaled to open up clogged airways. When CF affects the digestive system, the body does not absorb enough nutrients. Therefore, people with CF may need to eat an enriched diet and take both replacement vitamins and enzymes.

Although highly informative and successfully applied in basic as well as epidemiology studies, most of those methods are tedious and technically complex, in addition, it is sometimes difficult to employ these methods routinely in the clinical context, when the most important requirements are quality of service, speed accuracy, and low cost.

The detection of the ΔF508 mutation of the Cystic Fibrosis gene using the PCR based micro-cantilevers as a sensor can be divided into several procedures:

1. Cleaning the gold micro-cantilever

2. Immobilization of the detection probe to the surface of the micro-cantilever (programming of the micro-cantilever chip).

3. DNA isolation from the biological sample (in this example three patient samples).

4. Designing PCR primers for either single reactions or multiplex reactions.

5. The reaction step involving simultaneously PCR reaction probe hybridization and a 3′ extension reaction.

6. Measuring the bending of the micro-cantilever due to specific extension of the probe on the surface of the micro-cantilevers.

TABLE II
Hybridization probes and PCR primers

| | |
|---|---|
| Primer 1_{CF} | 5′-AAGCAAGAATATAAGACATTGG-3′ (sense) |
| Primer 2_{CF} | 5′-CTATATTCATCATAGGAAACAC-3′ |
| | (antisence) |
| PROBE_{wCF} | 5′DMT-S-(CH2)₁₂- |
| | CCATTAAAGAAAATATCATCTT-3′ |
| PROBE_{ΔCF} | 5′DMT-S-(CH2)₁₂- |
| | GCACCATTAAAGAAAATATCATCGG-3′ |

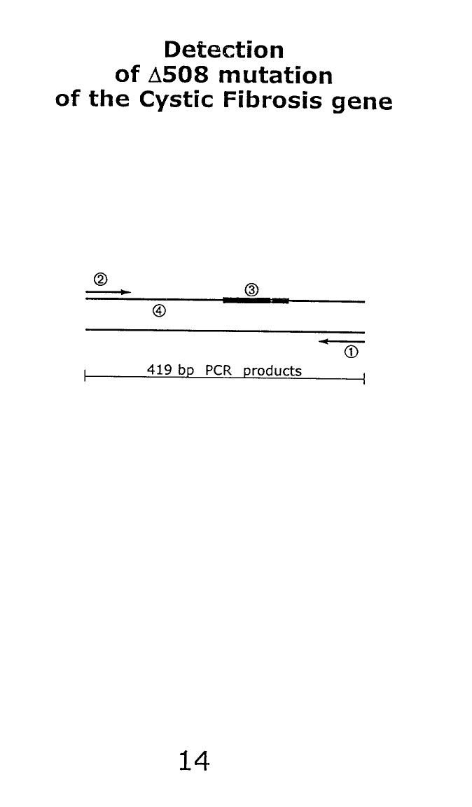

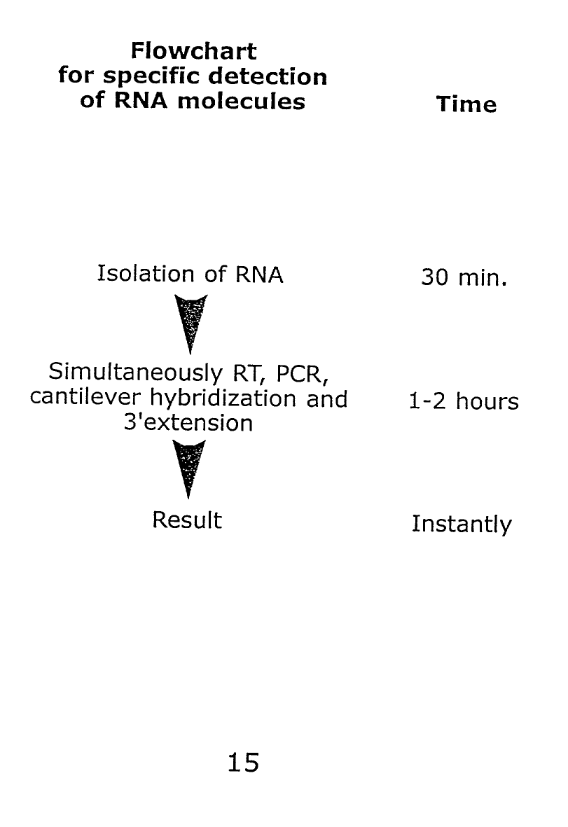

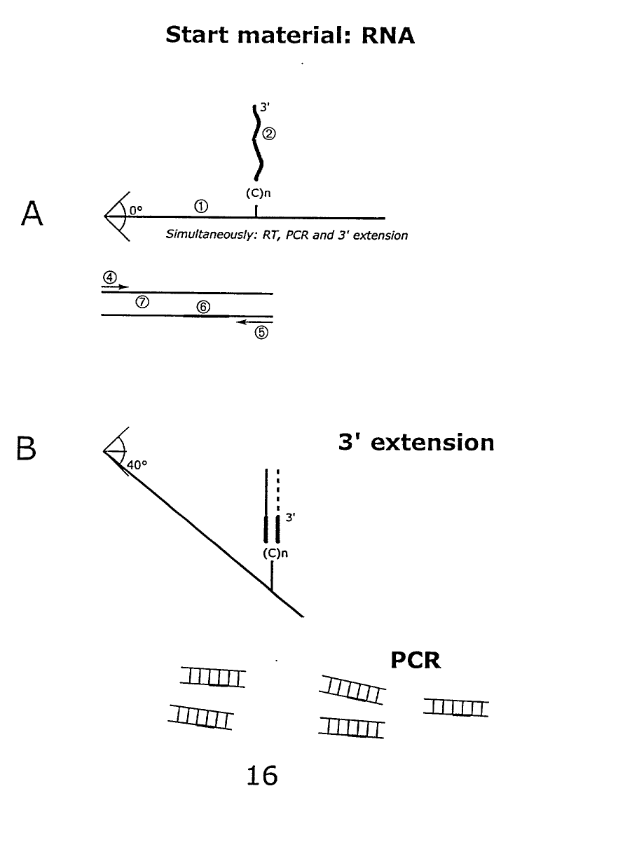

Both probes are located in close distance to PCR CFₚᵣᵢₘₑᵣ 2 as illustrated in FIG. 14. The primers for amplifying the CF gene fragment (419 bp) are illustrated at FIG. 14, and had the following and are both 22 mer and have A/T=15 and G/C=7.

The PCR reaction is performed under a specific temperature cycle profile, however the measurement of the micro-cantilever are taking place at 20 ° C.

Human genomic DNA was isolated from normal (sample 1), F508 del heterozygous subjects (sample 2) and homozygous patients (sample 3) using either Qiagen blood DNA isolation kit alternative 4 μl of whole blood was heated at 95° C. for 5 minutes and cooled at 30° C., after repeating this cycle 3 times, 46 μl of 1×PCR reaction mixture was added to a final volume of 50 μl (Ree DC et al. Thromb Haemost 75:520-526, 1996). The final 1×PCR solution included the genomic DNA, primers, dNTP, MgCl₂, Taq Gold polymerase, and buffer as standard concentrations as described in the user manual for PCR, Perkin Elmer.

Approx. x pl of the 50 μl reaction mixture was injected into the micro-cantilever via a flow pumping device. The reaction chamber was closed containing approx. x μl of reaction mixture. The micro-cantilever was placed on a Hybead thermo cycler equipped with a flat thermo controlled surface. The 30 cycles used were as follows: Denaturation, 30 see, 94° C.; annealing 30 see, 60 C.; and elongation, 1 minute, 72° C. After 30 cycles the temperature was set to 20° C. and the surface stress changes was measured for the two channels having either the wCF DNA probe or the ΔCF DNA probe immobilized. As shown in table III its was clearly a significant different between the three patient samples. The data obtained clearly shows that the _{w}CF DNA probe gave the biggest stress response in sample 1, next best stress response in sample 2 and the lowest stress response in sample 3 as expected. In contrary the ΔCF DNA probe gave the best stress response in sample 3, next best in sample 2 and the lowest stress response in sample 1 also as should be expected.

Results

As illustrated in table is was possible to detect the difference between normal, heterozygous and homozygous the ΔF508 mutation of the Cystic Fibrosis gene using the PCR based micro-cantilevers as a sensor.

TABLE III
unit N/m

| Patient samples No | _{w}CF gene | _{Δ}CF gene |
|---|---|---|
| 1. Normal CF gene | 5.1 | 0.4 |
| 2. Heterozygous _{ΔCF} CF | 3.1 | 2.9 |
| 3. Homozygous _{ΔCF} CF gene | 0.3 | 4.7 |

### Example 3

Detection of Interleukin 6 mRNA level between the ages group 20-35 year, 36-59 years and 60-70 years using micro-cantilever technology.

It's has been reported (Jolanta Musliwska et al. Mechanism of Ageing and Developement 100, 313-328, 1998) that the level of interleukin 6 (IL6) mRNA increases during the lifetime of a normal healthy person.

We use the PCR based micro-cantilevers as a sensor for detecting the IL6 mRNA level in 6 healthy Danish man in the ages 20-70 years old. We used 2 volunteers in each group (20-35 years group; the 36-59 years and the 60-70 years group).

The process can ne divided into several procedures:

1. Cleaning the gold micro-cantilever

2. Immobilization of the detection probe to the surface of the micro-cantilever (programming of the micro-cantilever chip).

3. RNA isolation from the biological sample (in this example 6 blood samples from healthy Danish men).

4. Designing PCR primers for either single reactions or multiplex reactions.

5. The reaction step involving simultaneously PCR reaction, probe hybridization and a 3′ extension reaction.

6. Measuring the bending of the micro-cantilever due to specific extension of the probe on the surface of the micro-cantilevers.

The cleaning of the gold micro-cantilever was performed as described in example 1. The quantitative analysis by RT-PCR can be difficult because of the exponential nature of PCR. A small variation during the assay might yield a marked change in the amount of the final products. The use of internal standards is therefor desirable in quantitative RT-PCR analysis to correct variations in RT-PCR as well as product detection step (micro-cantilever detection). An ideal endogenous standard would be a transcript in which the expression is constant during the cell cycle, between cell types or in response to external stimuli. A housekeeping gene GAPD that is transcribed constitutively in most cell types and tissue has been commonly used as an invariant control.

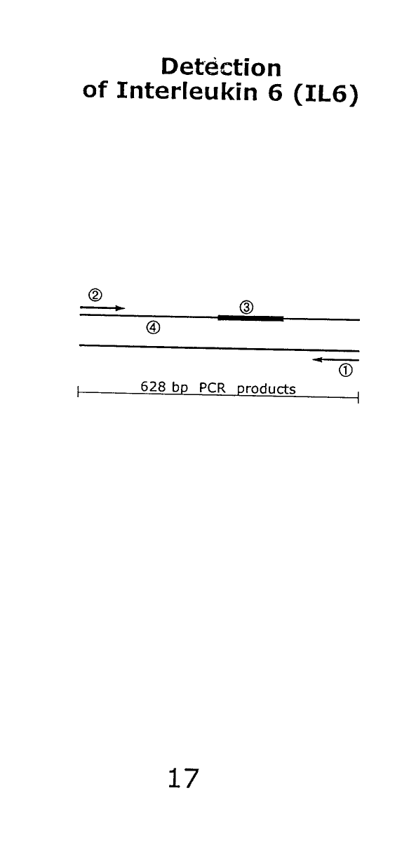

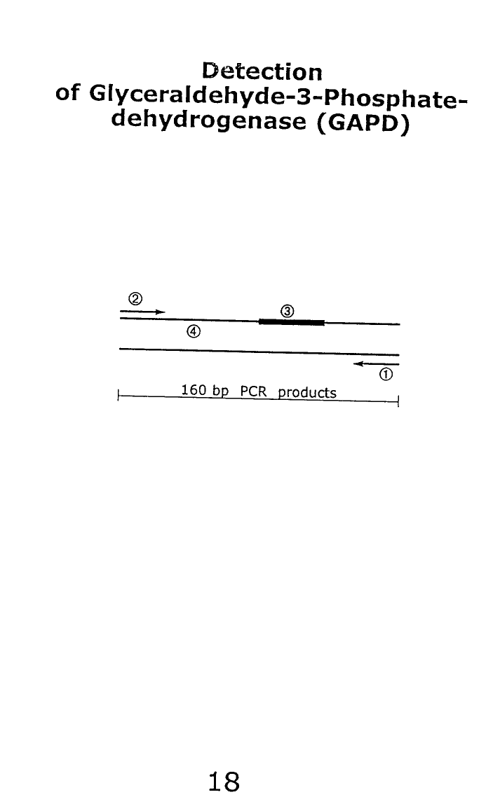

TABLE IV
Hybridization probes and PCR primers, both probes
are located in close distance to PCR Primer 2_{IL6}
and Primer 2_{GAPD} as illustrated in FIG. 17 and 18.

| | |
|---|---|
| PROBE_{IL6} | 5′DMT-S-(CH2)₁₂- |
| | CTGCGCAGCTTTAAGGAGTTCC-3′ |
| PROBE_{GAP} | 5′DMT-S-(CH2)₁₂- |
| _{D} | CGCTGGGGCTGGCATTGCCCTC-3′ |
| Primer | 5′-CATCAAGAAGGTGGTGAAGC-3′ (sense) |
| 1_{GAPD} | |
| Primer | 5′-GAGCTTGACAAAGTGGTCGT-3′ (antisense) |
| 2_{GAPD} | |
| Primer 1_{IL6} | 5′-ATGAACTCCTTCTCCACAAGCGC-3′ (sense) |
| Primer 2_{IL6} | 5′-GAAGAGCCCTCAGGCTGGACTG-3′ antisense) |

The IL6 probe and the GAPD probe are immobilized as described in example 1. The RNA was isolated from the biological sample in this example from 6 blood samples from healthy Danish men). The RNA from the Human peripheral blood cells was obtained as described in the Qiagen blood RNA isolation kit.

The reaction step involving simultaneously PCR reaction, probe hybridization and finally a 3′ extension reaction.

In summery 0.5 μg total RNA was added together with the specific PCR amplifications primers (total of 4 primers, 2 sets) and a poly dT₁₈ primer to the Titan One Tube RT-PCR System, the concentration of the various components are according to manufacture, in short summery components of the Titan One Tube RT-PCR System allow completion of RT-PCR in a one-step reaction. The system includes the following components: Enzyme mix containing AMV Reverse Transcriptase (for reverse transcription) and the Expand High Fidelity PCR System (Taq/Pwo enzyme blend, for PCR) Roche Biosystems. All components was mixed and added to the closed micro-cantilever systems, approx. x μl reaction volume. The micro-cantilever was placed on a Hybead thermo cycler equipped with a flat thermo controlled surface. The 30 cycles used were as follows: Denaturation, 30 sec, 94° C.; annealing 60 sec, 60° C.; and elongation, 1 minute, 72° C.

After 30 cycles the temperature was set to 20° C. and the surface stress changes was measured for the two channels having either the IL6 DNA probe or the GAPD DNA probe immobilized.

Results

As shown in table V its was clearly a significant different between the six patient samples. The data obtained clearly shows that the IL6 DNA probe gave the biggest stress response in the ages group 60-70 years, next best stress response in ages group 36-59 years and the lowest stress response in the ages group 20-35 year. Those results are in agreement with the results previous described by Jolanta Musliwska.

TABLE V
Units N/m

| | Channel 1 | Channel 2 | PROBE_{IL6}/ |
|---|---|---|---|
| Volunteers | PROBE_{IL6} | PROBE_{GAPD} | PROBE_{GAPD} |
| 1 20-35 year | 2.1 | 2.1 | 1 |
| 2 | 2.6 | 2.7 | 0.96 |
| 1 36-59 years | 4.1 | 1.9 | 2.16 |
| 2 | 3.9 | 2.4 | 1.63 |
| 1 60-70 years | 5.6 | 1.8 | 3.11 |
| 2 | 6.1 | 1.7 | 3.59 |

### Example 3

Detection of Herpes Simplex Virus DNA using micro-cantilever technology. Viral load is becoming the diagnostic and prognostic analysis of choice for viral disease. Unlike antibody-based methods of detection viral infection, nucleic acid analysis provides quantitative information about viral levels. In Herpes Simplex Virus (HSV) infection, viral load is a key indicator of disease progression and a valuable method for monitoring the success rate of different therapies. Viral load is therefore an good example of the used of micro-cantilever technology.

The detection of the of Herpes Simplex Virus DNA using the PCR based micro-cantilevers as a sensor; the process can be divided into several procedures:

1. Cleaning the gold micro-cantilever

2. Immobilization of the detection probe to the surface of the micro-cantilever (programming of the micro-cantilever chip).

3. DNA isolation from the biological sample (in this example 2 blood samples from a healthy Danish man and a patient infected with the of Herpes Simplex Virus).

4. Designing PCR primers for either single reactions or multiplex reactions.

5. The reaction step involving simultaneously PCR reaction, probe hybridization and a 3′ extension reaction.

6. Measuring the bending of the micro-cantilever due to specific extension of the probe on the surface of the micro-cantilevers.

The cleaning of the gold micro-cantilever was performed as described in example 1.

TABLE VI
Hybridization probes and PCR primers. The PCR
primer give a 179 bp fragment of the HSV
polymerase gene, the HSV probe are located in
close distance to Primer 2_{HSV}

| | |
|---|---|
| PROBE_{HSV} | 5′DMT-S-(CH2)₁₂- |
| | CAGCAAGATAAAGGTGAACGGC-3′ |
| Primer 1_{HSV} | 5′-ATCAACTTCGACTGGCCCTTC-3′ (sense) |
| Primer 2_{HSV} | 5′-CCGTACATGTCGATGTTCACC-3′ (antisense) |

Human genomic DNA was isolated from non infected human DNA (sample 1) and a HSV infected human DNA (sample 2) using either Qiagen blood DNA isolation kit. The final 1×PCR solution included the genomic DNA, primers, dNTP, MgCl₂, Taq Gold polymerase, and buffer as standard concentrations as described in the user manual for PCR, Perkin Elmer.

Approx. x μl of the 50 μl reaction mixture was rejected into the micro-cantilever via a flow pumping device. The reaction chamber was closed containing approx. 1 μl of reaction mixture. The micro-cantilever was placed on a Hybead thermo cycler equipped with a flat thermo controlled surface. The 30 cycles used were as follows: Denaturation, 45 sec, 95° C.; annealing 45 sec, 62° C.; and elongation, 1 minute, 72° C. After 35 cycles the temperature was set to 20° C. and the surface stress changes was measured for in one channel, having the PROBEH_{HSV} immobilized.

Result

The data shown in table VII clearly shows that the PROBEH_{HSV} gave the biggest stress response in sample 2 (infected DNA) compare to sample 1 (non infected DNA).

TABLE VII
unit N/M

| Patient samples No | PROBE_{HSV} |
|---|---|
| 1. Non infected DNA | 0.4 |
| 2. HSV infected DNA | 2.1 |

What is claimed is:

1. A method for determining the presence or absence of a target nucleic acid in a test sample comprising:
contacting a target nucleic acid comprising first and second adjacent regions with a piezoelectric biosensor comprising a microsensor having a surface comprising an immobilized probe nucleic acid which hybridizes to said first region of said test nucleic acid to form a hybridization complex, wherein said first region of said target nucleic acid is double stranded and said adjacent second region of said target nucleic acid is single stranded in said hybridization complex;
extending the probe nucleic acid in said hybridization complex using said second region in said test nucleic acid as template; and
measuring a parameter of said piezoelectric biosensor which provides an indication of whether or not said target nucleic acid is present in said test sample.

2. The method of claim 1 wherein extension of said probe nucleic acid is dependent upon base pair matches or mismatches with one or more opposing nucleotides in said first or said second region.

3. The method of claim 2 wherein said base pair match or mismatch is not the terminal nucleotide of said probe nucleic acid.

4. The method of claim 1 wherein said piezoelectric biosensor comprises a piezoelectric element and said parameter is resistance, current or voltage.

5. The method of claim 1 wherein said piezoelectric biosensor comprises a piezoelectric element and said microsensor undergoes a change in resonant oscillation frequency or amplitude upon probe extension as measured by a charge in resistance, current or voltage of said piezoelectric element.

6. The method of claim 1 further comprising amplifying said target nucleic acid.

7. The method of claim 5 wherein said amplifying is by PCR or LCR and said amplification occurs simultaneously with said contacting.

8. The method of claim 3 wherein the presence of a base pair match or mismatch at said terminal nucleotide in said hybridization complex is indicative of the substitution, insertion or deletion of one or more nucleotides in said test nucleic acid as compared to said probe nucleic acid.

9. The method of claim 1 wherein the measurement of said parameter provides an indication of the concentration of said target nucleic acid in said test sample.

10. A devise for detecting the presence or absence of a target nucleic acid comprising:
a piezoelectric element;
a microsensor in mechanical communication with said piezoelectric element, said microsensor having a surface comprising an immobilized probe nucleic acid;
an oscillator in mechanical communication with said microsensor;
an oscillator controller in mechanical communication with said piezoelectric element;
a measurement device for measuring oscillation amplitude or resonance frequency of said microsensor in response to a parameter of said piezoelectric element.
